(19) United States
(12) Patent Application Publication
(10) Pub. No.: US 2013/0123355 A1
(43) Pub. Date: May 16, 2013

(54) Fatty acids and metal ions compositions and uses thereof

(71) Applicant: Maninder Chopra, Fort Myers, FL (US)

(72) Inventor: Maninder Chopra, Fort Myers, FL (US)

(21) Appl. No.: 13/674,832

(22) Filed: Nov. 12, 2012

Related U.S. Application Data
(60) Provisional application No. 61/559,027, filed on Nov. 11, 2011.

(51) Int. Cl. A61K 31/202

(52) U.S. Cl. CPC A61K 31/202
USPC 514/494; 514/560

(57) ABSTRACT

Compositions comprising various fatty acids and their variants such as DHA, DHA-lysoPC, and EPA combined with neuro-therapeutic metal ions such as magnesium (Mg), zinc (Zn) or lithium (Li), capable of traversing the blood-brain barrier (“BBB), and blood nerve barrier (“BNB”) and methods of use of such compositions to provide therapeutic concentrations of Mg, Zn, or Li in cerebrospinal fluid over a desired period of time to a subject presenting symptoms of Mg, Zn, or Li deficiency or disease symptoms which are alleviated by supplementation of such ions are provided These symptoms may include, but are not limited to, mild cognitive impairment, bipolar disorder, stroke, and pain.

### CROSS-REFERENCE TO RELATED APPLICATIONS

This application claims priority to a provisional application entitled “Fatty acids metal ions compositions and methods thereof,” Ser. No. 61,559,027, filed Nov. 11, 2011, which is incorporated in its entirety by reference herein.

### BACKGROUND

Magnesium, the second most abundant intracellular cation, plays a crucial role in many physiological functions. These include maintenance of normal muscle and nerve function, blood pressure, healthy immune system, bone strength, and heart rhythm, and regulation of blood sugar levels. At the molecular level, magnesium is involved in more than 300 enzymatic reactions in the body thus regulating some of the most important biological activities in humans. At the cellular level, in addition to being involved in the homeostasis of other minerals such as sodium, potassium and calcium, magnesium also plays an important role in all reactions involving the formation and utilization of adenosine triphosphate (ATP), the energy currency in the living cells.

One function of magnesium in the brain is to modulate the voltage-dependent block of NMDA receptors. These receptors are involved in synaptic plasticity, which in turn is involved in a number of neuronal functions such as cognitive functions including learning and memory. Magnesium has been reported to play a role in the regulation of synaptic plasticity, the mechanism by which neural circuits are organized during early development and memory traces are encoded and stored throughout life. The degree of synaptic plasticity depends on both the plasticity of synaptic connection and the number of these connections. Loss of synaptic plasticity is associated with an aging or diseased brain. For example, the forgetfulness that is common in older people is generally associated with loss of synaptic plasticity in the hippocampus, a brain region associated with short-term memory. In addition, loss of synaptic plasticity may lead to cognitive impairment or various dementias including Alzheimer's disease (AD). AD patients have significantly lower levels of magnesium in their cerebrospinal fluid as compared to non-AD humans of the same age group. Mammals, including companion animals such as dogs and cats, are known to have old age syndrome characterized by general lethargy, decreased metabolism and cognitive deficits. The mechanism of cognitive decline is likely to be shared with those in humans.

Depression, especially major and suicidal, has been linked to magnesium deficiency disorder. There is a long history of the use of magnesium to treat depression and other mental health issues. For example, magnesium sulfate (10% elemental magnesium) injected in doses of one to two cc. of a 25% or 50% solution helped agitated depressed patients sleep from 4 to 6 hrs.

Due to its role in blocking the NMDA receptor and thus inhibiting calcium entry into the cell, magnesium along with the NMDA receptor is thought to be involved in the regulation of pain. In several clinical trials, magnesium alleviates postoperative pain intensity and decreases analgesic requirements.

Given the importance of magnesium in human health, many people may not consume enough magnesium in their diets. Studies have reported that more than 70% of diets in the US and the West contain less than the recommended intake of 400 mg of magnesium per day and up to 20% of diets contain less than half of this amount. Magnesium deficiency symptoms may include loss of appetite, fatigue, nausea, vomiting, seizures, abnormal heart rhythms and other cardiovascular disease, and diabetes. In addition, due to its role as an essential cofactor for enzymes in the brain, magnesium deficiency in the cerebrospinal fluid has been linked to major depression, adjustment disorder, personality changes, and anxiety.

Approximately 33% of dietary magnesium is absorbed, the majority by the small intestine and a small amount by the large intestine. The kidneys through regulating magnesium excretion maintain magnesium balance in the bloodstream.

Commercially available magnesium supplements include magnesium oxide, magnesium hydroxide, magnesium sulfate, magnesium chelate compounds, and various organic magnesium salts, for example, of gluconic acid, citric acid, and lactic acid. Although these supplements may be high in elemental magnesium, they have very low magnesium systemic bioavailability, or capacity for absorption in the human body. Further, most such magnesium salts have poor penetration through BBB, thus no significant CNS benefits from such magnesium compositions are observed.

According to the National Institute of Health U.S. guidelines, the recommended daily intake of magnesium for a male adult is 400-420 mg/day, and for a female adult 310-320 mg/day. Foods such as green vegetables, for example spinach, some legumes (beans and peas), nuts and seeds, chocolate, milk, and whole, unrefined grains are a good source of magnesium. Despite the ready availability of these foods, there is not enough consumption to satisfy the daily nutritional requirements. Also, current dietary habits in the U.S. and generally in the West lean more towards processed foods, which tend to contain less magnesium. Given the role of magnesium in human health, an oral magnesium supplement that offers high bioavailability with fewer side effects is highly recommended and desired.

Methods and apparatus for increasing magnesium levels in human body tissues have been previously described. For example, intravenous administration of magnesium sulfate to patients has been used for the treatment of arrhythmia. In addition dietary supplements such as magnesium oxide, magnesium hydroxide and magnesium carbonate have been used to provide magnesium to human body. Although, these compounds may increase magnesium levels in the human body tissues, they are mostly insoluble in the gastrointestinal tract, and hence, not readily absorbed by the gastrointestinal membrane. These compounds do not raise the brain levels of magnesium by significant levels. In one study, even an intravenous infusion of magnesium sulfate for 5 days, which increased the blood levels of magnesium by 3-fold failed to increase Mg levels in the brain. Magnesium threonate (MgT) has been proposed to supply means for providing magnesium to the human body, particularly across the BBB, and is claimed to address the poor absorption and side effects associated with other magnesium compounds. However, the efficacy of MgT in providing magnesium in human subjects is not clear since the studies, so far, have been conducted in rats as model system, and may or may not translate to humans.

Similar to magnesium, zinc is the second most prevalent essential trace element in living organisms, and is required for several molecular targets, such as enzymes including metallothioneins, several voltage-gated ion-channels, neurotransmitter transporters and receptors. Zinc is particularly abundant in the CNS where it plays an important role in synaptic transmission, leading to a variety of roles in brain functions such as motor control, neurological disorders including pain, depression, cognitive dysfunctions such as observed in dementias including Alzheimer's. One possible mechanism by which zinc alleviates pain is through high-affinity binding to the NMDA receptor NR2A subunit.

Lithium supplementation, generally lithium carbonate, has been used to treat manic-depressive disorder (bipolar disorder) where it stabilizes the mood and reduces incidents of extremes in behavior such as exaggerated feelings of well-being, irritability, anxiousness, rapid or loud speech, and aggressive behavior by restoring the balance of neurotransmitters in the brain. Lithium has also been used to increase the efficacy of antidepressant medications when the later have not worked fully in treating depression. Lithium therapy through oral route results in lithium distribution throughout the CNS where it interacts with various neurotransmitters and receptors to decrease norepinephrine release and increase serotonin synthesis. However, oral supplementation of lithium is associated with considerably side effects such as nausea, diarrhea, shaking of hands, dizziness, seizures, confusion, slurred speech, polydipsia, increase in the amount of urine, weight gain, hypothyroidism, and downbeat nystagmus. Patients undergoing chronic therapy with lithium may accumulate high levels of lithium resulting in toxicity manifested as nausea, emesis, diarrhea, asthenia, ataxia, confusion, lethargy, polyuria, seizures, coma, coarse tremor, muscle twitching, convulsions and renal failure.

To treat neurological disorders due to Mg, Zn, or Li deficiencies, current therapeutic strategies involve administering compounds of Mg, Zn or Li, primarily by oral route, to increase the levels of Mg, Zn or Li in the blood and thereby in the brain. However, the dose required to raise the levels of these divalent metal ions in brain to levels required for efficacy is considerably higher due to a barrier system; the blood-brain barrier that separates the blood from ISF. Moreover, the levels of Mg, Zn, or Li must be tightly regulated since higher levels of these cations are associated with various side effects. Thus there is a need in the art to deliver neuro-therapeutic cations such as, but not limited to, Mg, Zn, and Li to the brain.

Previous studies have been directed to transporting therapeutic, pharmaceutical agents across the BBB. For example, gama-aminobutyric acid (GABA) uptake into the brain can be enhanced by 100 fold if GABA is linked to 1,2-dilinolnoyl glycerol, a molecule that is normally transported across the BBB. U.S. Pat. No. 5,604,198, refers to a method to enhance the ability of neurologically active compounds to penetrate the blood nerve barrier (BNB) or BBB by conjugating to a carrier molecule that has been shown to have a substantial permeability coefficient across the BNB or BBB, such as hemoglobin, lysozyme, cytochrome c, ceruloplasmin, calmodulin, ubiquitin or substance P. In addition, other molecules such as, cholesterol, dexamethasone have been identified as carriers to transport across the BBB. In yet another example, magnesium was supplied in the form of threonate salt with claims to cross BBB with efficiency better than other magnesium compositions in animal models.

Polysaturated fatty acids are transported across BBB into the brain, where they play a variety of important roles. This includes docohexaenoic acid (DHA, 22:6n-3) and eicosapentaenoic acids (EPA), which are accumulated in the brain from blood. DHA is a naturally occurring, 22-carbon chain un-branched fatty acid of the omega-3 family derived from the precursor linolenic acid, an essential fatty acid. DHA is the most abundant fatty acid in the brain, retina and sperm where it is mainly esterified in membrane phospholipids. DHA is important for brain development and its deficiency is linked to cognitive decline. DHA supplementation has been proposed as a preventive therapy against Alzheimer's disease as a neuro-protective agent to prevent or delay development of Alzheimer's disease and cognitive decline associated with Alzheimer's and other dementias or old age.

DHA has been used previously as a carrier to help deliver drugs across the blood brain barrier. For example, U.S. Pat. No. 6,258,836 refers to formation of a pro-drug from a fatty acid carrier, preferably DHA and a neuro-active drug, preferably dopamine. The pro-drug passes through the BBB, having a brain penetration index of at least two times of the drug alone. Similarly, compositions consisting of DHA conjugates covalently linked with neuro-active drugs have been disclosed, such as, choline (U.S. Pat. No. 6,153,653), clozapine (U.S. Pat. No. 6,197,764), and anti-psychotic agents (U.S. Pat. No. 5,955,459), to increase brain penetration of these pro-drugs. The mechanism by which DHA helps transport conjugates across the BBB is largely not well understood, but most likely involves diffusion across the BBB due to their physicochemical properties, including high level of lipid solubility and relatively small molecular weight (328 kDa). DHA is modified in a number of related compounds, which are thought to enhance the accumulation in the brain and other tissues. Some of these forms include triglycerides, phosphatidylcholines and lysophosphatidylcholine. The lysophosphatidylcholine-DHA along with non-esterified DHA has been suggested to be main means to transport DHA moiety to the brain. DHA is also known to be oxidized into various derivatives such as resolvins and neuroprotectin D1, which may have bioactive effects as anti-inflammatory and neuroprotective agents.

EPA is 20-carbon chain un-branched fatty acid of the omega-3 family derived from the precursor linolenic acid, and is among essential fatty acids accumulated in the brain, presumably by mechanisms similar to those of DHA. In fact, the permeability of DHA and EPA is similar in animal models. EPA has been shown to have efficacy in a number of psychiatric disorders, including depression and schizophrenia

### SUMMARY

Cations such as magnesium or zinc play an important role in brain health, function, and performance. In addition, many cations, such as lithium, have been therapeutically used for certain brain disorders, such as bipolar syndrome. The presence of a tightly regulated physiological barrier prevents many substances in the blood to enter brain. Some cations such as magnesium, zinc, and lithium are prevented to freely cross the said ‘blood-brain barrier’ (BBB). Similar barrier, known as blood-nerve barrier (BNB) exist between blood and nerves. Due to general lack of suitable delivery methods for supplying these cations across the blood brain barrier, attempts to provide adequate levels of these cations for therapeutic purposes have been largely unsuccessful. Frequently, such attempts have been focused on very high level of dosing to compensate for relative impermeability of these cations imposed by the BBB or BNB. Aspects discussed herein provide methods to deliver magnesium, lithium, or zinc from blood through the BBB into the brain.

Other aspects provide compositions and methods for enhancing permeability of the desired cations across the BBB, thus allowing higher levels of such ions in the brain, as measurable in the cerebrospinal fluid. In another aspect, compositions are contemplated to have an ability to enhance levels of desired cations in the brain to level sufficient for restoring various brain functions. It is anticipated that, in some aspects, an increase in the desired cations in the brain by will interact with molecular targets that are involved in various disease processes, and hence will provide a therapeutic benefit for individuals suffering with those various diseases.

By providing a defined benefit to the person in need of the desired cations in the brain, the aspects described herein can act additively or synergistically with other therapeutics agents for the same indication, thus allowing a lower amount of the said other medicine to be administered, thus mitigating or reducing the side effects of the said other medicine. The synergistic effects will lead to an increased efficacy to a level not achievable by the other medicine alone.

Essential fatty acids, including DHA, and EPA are known to be relatively permeable across the BBB, since they are known to be required for various brain functions. Aspects described herein employ these essential fatty acids not only as carriers for metal ions to cross BBB and BNB, but also to provide therapeutic effects on their own. Thus, aspects described herein utilize a role for the carrier molecules synergistic with the metal ions being transported into the nervous system.

These compositions will also provide therapeutic benefits to mammals, in need thereof, which share similar physiological and disease mechanisms as humans. For instance, dogs are known to have ‘old-age syndrome’ characterized by cognitive decline similar to that observed in aged humans. Thus, various compositions contemplated herein would be expected to have therapeutic effects on these animals.

This object and others are met by one or more aspects directed to compositions comprising: a divalent metal ion, for example, magnesium, zinc or a monovalent metal ion, for example, lithium; complexed to a fatty acid, for example, and preferably, DHA, DHA-lysoPC, EPA or any variant or combination thereof. Various means of complexing the said metal ions with the said fatty acids comprise a variety of chemical bonds (e.g., ionic, covalent, and pi bonds).

One aspect provides compositions comprising: a mixture of fatty acids, such as seen in fish oil (DHA+EPA) complexed to divalent metal ions such as magnesium, zinc or a monovalent metal ion such as lithium.

In additional aspects, the active agent is magnesium, zinc, or lithium complexed with fatty acids.

In other aspects, the active agent is magnesium, zinc, or lithium complexed with DHA.

In other aspects, the active agent is magnesium, zinc, or lithium complexed with DHA-LysoPC.

In other preferred aspects, the active agent is magnesium, zinc, or lithium complexed with EPA and its derivatives.

In one aspect, at least a portion of cations selected from a group consisting of: Mg, Zn and Li are complexed through ionic bond with fatty acids selected from a group consisting of: DHA, DHA-lysoPC and EPA.

In another aspect, at least a portion of cations selected from a group consisting of: Mg, Zn and Li are complexed covalently with fatty acids selected from a group consisting of: DHA, DHA-lysoPC and EPA.

In yet another aspect, at least a portion of cations selected from a group consisting of: Mg, Zn and Li are complexed through pi bonds with fatty acids selected from a group consisting of: DHA, DHA-lysoPC and EPA.

In further aspects, the active agent is magnesium, zinc, or lithium complexed with fatty acids, such as DHA, DHA-lysoPC, or EPA in the presence of one or more antioxidants, to prevent oxidation of fatty acids.

In additional aspects, the active agent is magnesium, zinc, or lithium complexed with DHA in the presence of one or more antioxidants.

In other aspects, the active agent is magnesium, zinc, or lithium complexed with DHA-lysoPC in the presence of one or more antioxidants.

In some aspects, the active agent is magnesium, zinc, or lithium complexed with EPA in the presence of one or more antioxidants. Other aspects further comprise drugs targeting the brain.

Other aspects are directed to methods of treating a medical condition by providing an active agent through oral intake or other suitable modes of administration, wherein the active agent is either a divalent metal ion, for example, Mg, Zn or a monovalent metal ion, for example, Li; complexed to DHA, DHA-lysoPC, EPA, or any variant or combination thereof. The oral dosage form can be in the form of a capsule, tablet, gel, liquid or any other known format. In addition, the medicament could be provided through other routes of administration, such as enteral, intravenous, intramuscular, subcutaneous, intrathecal, epidural, intracerebroventicular, nasal, rectal, vaginal, and transdermal routes.

Other aspects provide methods of treating disease symptoms arising out of deficiency of the said metal ion. In other aspects, the metal ions levels in the brain are raised above the normal range to provide therapeutic benefits from a disease by counteracting some other biochemical malfunctioning responsible for the disease.

Another aspect provides a method for enhancing, and/or maintaining cognitive function, such as in various psychiatric and neurological disorders, including Schizophrenia, unipolar and bipolar depression, dementias including Alzheimer's disease, and short-term cognition impairment following post-surgical anesthesia. The method comprises the step of administering to a subject an amount of Mg-DHA, Mg-DHA-lysoPC, Mg-EPA or any variant or combination thereof, in sufficient/effective to increase physiological concentration of magnesium to a level and duration, which is sufficient for a therapeutic effect. In some instances, the concentration of magnesium is measured under a fasting condition since it gives more consistent readings. In some instances, the cognitive function is short-term memory or long-term memory. The cognitive function sought to be restored either completely or partially is that observed in a number of psychiatric conditions, such as dementias including Alzheimer's disease, Schizophrenia, depression, anxiety, or that seen in post-intensive care unit treated patients as a part of post-operative symptoms.

Magnesium ion modulates NMDA receptor, thus an increase in Mg²⁺ in the brain may effect various downstream functions of the NMDA receptor, which in turn may effect various diseases including acute and chronic decline in cognitive functions, pain, depression, stroke etc. It is anticipated that an increase in magnesium in the cerebrospinal fluid will provide a therapeutic benefit for the above-mentioned diseases. Similarly, magnesium affects other molecular components of the brain, and by providing increased levels of magnesium in the brain, aspects described herein are anticipated to provide therapeutic benefits by modulating its downstream molecular targets that are involved in various disease conditions.

By providing a defined benefit to the person in need of the increased magnesium in the brain, aspects described herein can act additively or synergistically with other therapeutics agents for the same indication, thus allowing a lower amount of the other medicine to be administered, thus mitigating or reducing the side effects of the other medicine.

A composition, and/or a method described herein may be useful for enhancing or maintaining cognition, learning, memory function or treating subjects for Alzheimer's disease, depression, pain, including neuropathic pain, recovery from stroke, and bipolar disorder, eclampsia, epileptic seizures, post-operative shivering, ADHD, merely by way of example.

Another aspect provides a method of ameliorating pain in a subject in need thereof, comprising the step of administering to said subject an amount of Mg-DHA, Mg-DHA-lysoPC, Mg-EPA or any variant or combination thereof, in an amount effective to increase physiological concentration of magnesium to a level and duration sufficient to provide therapeutic effect. In some instances, the concentration of magnesium is measured under physiological conditions. In other instances, the physiological concentration of magnesium is serum concentration, plasma concentration, or cerebrospinal fluid concentration. In other instances, the magnesium compositions are administered as an adjuvant to postoperative and perioperative analgesia to have more favorable effects on pain intensity and analgesic requirements, and to manage hypomagnesaemia, and to decrease the incidence of post-operative shivering.

Aspects described herein provide a method of use of Mg-DHA, Mg-DHA-lysoPC, Mg-EPA, magnesium compounds of fatty acids, or a combination thereof for recovery of patients who have suffered from stroke.

Zinc is known to modulate NMDA receptor, and is known to be the mechanism for alleviating pain. Like Magnesium, elevating Zn levels would provide similar benefits related to NMDA receptor function, such as pain, depression, and cognition. Aspects described herein provide methods of therapeutic or prophylactic treatment of zinc related disorders, comprising administering to a said subject, in need thereof, a composition of Zn-DHA, Zn-DHA-lysoPC, Zn-EPA or any variant or combination thereof in dose(s) which leads to concentrations of zinc in the brain or CNS sufficient to provide therapeutic benefits on the disease.

In another aspect it is contemplated that Mg and Zn ions modulate the NMDA receptor at different intramolecular sites, and their combined use may lead to a synergistic effect. Such a combination dosing is contemplated to enable a desirable enhanced therapeutic efficacy. The said combination dosing may enable an optimal therapeutic efficacy, where a single agent does not reach that desired level. It is also contemplated that if both the agents on their own provide the desired efficacy at a certain dosing levels, the combination may allow a smaller dosing of one or both compositions, due to additive or synergistic interaction of each active ingredient.

Another aspect provides a method of treating bipolar disorder. The method comprises the step of administering to a subject in need for a therapeutic treatment of bipolar disorder, a composition of Li-DHA, Li-DHA-lysoPC, Li-EPA or any variant or combination thereof in dose(s) sufficient, which leads to concentration sufficient to provide therapeutic benefits on the disease. In other aspect, a composition of Li-DHA, Li-DHA-lysoPC, Li-EPA or any variant or combination thereof is administered to a subject in need for a therapeutic treatment of bipolar disorder, in dose(s) sufficient for therapeutic effect on the disease.

Another aspect provides a method for increasing the efficacy of depression medications to ameliorate depression. This method comprises the step of administering to a subject in need for a therapeutic or prophylactic treatment of depression, a composition of Zn-DHA, Zn-DHA-lysoPC, Zn-EPA or any variant or combination thereof and Mg-DHA, Mg-DHA-lysoPC, Mg-EPA or any variant or combination thereof, in dose(s) sufficient to provide therapeutic benefits on the disease. In other aspects, a composition of Zn-DHA, Zn-DHA-lysoPC, Zn-EPA or any variant or combination thereof and Mg-DHA, Mg-DHA-lysoPC, Mg-EPA or any variant or combination thereof is administered to a subject in need thereof.

### INCORPORATION BY REFERENCE

All publication and patent applications recited herein incorporated in their entirety by reference.

### DETAILED DESCRIPTION

While certain aspects have been shown and described herein, numerous variations, alternatives and substitutions will occur to those skilled in the art without departing from the invention.

### DEFINITIONS

As used herein, the term “patient” refers to animal species of mammalian origin, including humans.

As used herein, the term “active” refers to the component, ingredient, or constituent of the compositions of aspects described herein responsible for the intended therapeutic effect.

It must be noted that a word appearing herein in the singular encompasses its plural counterpart; a word appearing herein in the plural encompasses its singular counterpart, unless the context clearly states otherwise.

Unless specifically stated otherwise, all scientific and technical terms used herein have the same meaning as commonly understood by one of ordinary skill in the art to which this invention pertains. Although many methods and materials similar or equivalent to those described herein can also be used in the practice or testing of the present invention, the preferred methods and materials are now described. All publications mentioned herein are incorporated herein by reference.

As used herein, the term “cognition” may refer to a process of obtaining, organizing, understanding, processing, or using information, knowledge, or sensory inputs.

The term “pro-drug” as used herein refers to a metabolically labile derivative that is pharmacologically inactive in the parent form but that is rapidly metabolized in human or animal plasma to a pharmacologically active form. Examples of pro-drugs as used herein include but in no way are limited to, where applicable, ester derivatives of carboxylic acids and of hydroxyl containing moieties or amide derivatives of carboxylic acids, such esters or amides include, but are not limited to those formed from substituted or un-substituted natural or un-natural amino acids.

The term “compound” is used herein to refer to any specific chemical compound disclosed herein. Within its use in context, the term generally refers to a single compound, but in certain instances may also refer to stereoisomers and/or optical isomers (including enantiopure compounds, enantiomerically enriched compounds and racemic mixtures) of disclosed compounds.

The term “pharmaceutically acceptable carrier” as used herein means one or more compatible solid or liquid filler, dilutants or encapsulating substances that are suitable for administration to a human or other animal. The term “carrier” denotes an organic or inorganic ingredient, natural or synthetic, with which the active ingredient is combined to facilitate the application. The components of the pharmaceutical compositions are capable of being commingled with the molecules of the invention, and with each other, in a way so that there is no interaction that would substantially impair the desired pharmaceutical efficacy.

The term “providing” or “administering” as used herein include other method of dispensing the pharmaceutical composition, e.g. prescribing.

The term “effective amount” refers to the amount of a selected compound that is used within the context of its intended use to affect an intended result, for example, to modulate glutamatergic synaptic response. The precise amount used will vary depending upon the particular compound selected and its intended use, the age and weight of the subject, route of administration, and so forth, including the duration of its use, but may be easily determined by routine experimentation. In the case of the treatment of a condition or disease state, an effective amount is that amount which is used to effectively treat the particular condition or disease state.

The term “schizophrenia” is used to describe a condition which is a common type of psychosis, characterized by a disorder in the thinking processes, such as delusions and hallucinations, and extensive withdrawal of the individual's interest from other people and the outside world, and the investment of it in his or her own. Schizophrenia is now considered a group of mental disorders rather than a single entity, and distinction is made between reactive and process schizophrenias. As used herein, the term schizophrenia or “schizophreniform” embraces all types of schizophrenia, including ambulatory schizophrenia, catatonic schizophrenia, hebephrenic schizophrenia, latent schizophrenia, process schizophrenia, pseudoneurotic schizophrenia, reactive schizophrenia, simple schizophrenia, and related psychotic disorders which are similar to schizophrenia, but which are not necessarily diagnosed as schizophrenia per se. Schizophrenia and other psychotic disorders may be diagnosed using guidelines established in, for example, Diagnostic and Statistical Manual of Mental Disorders, Fourth Edition (DSM IV) Sections 293.81, 293.82, 295.10, 295.20, 295.30, 295.40, 295.60, 295.70, 295.90, 297.1, 297.3, 298.8.

The term bipolar describes a class of mood disorders characterized by episodes of abnormally elevated energy levels, cognition, and mood collectively referred as mania or, if milder, hypomania, followed by episodes of depression. These states are usually separated by periods of normal mood.

The term depression describes a category of mood disorders accompanied by period of sad, irritable mood exceeding normal sadness or grief. These feelings are characterized by a greater intensity and duration of an all-encompassing low mood and low self-esteem. This condition may also include changes in bodily functions such as crying spells, body aches, low libido, and problems with eating, or sleeping.

The term cognitive decline refers to a brain function syndrome involving the onset and evolution of deterioration in cognitive function beyond those expected in normal aging process. This condition is often a transitional stage between normal aging and dementia.

The terms Mg-DHA, Mg-EPA, and Mg-DHA-lysoPC refer to generic names for compositions comprising at least some Mg complexed with DHA, EPA, or DHA-lysoPC respectively.

The terms Li-DHA, Li-EPA, and Li-DHA-lysoPC refer to generic names for compositions comprising at least some Li complexed with DHA, EPA, or DHA-lysoPC respectively.

The terms Zn-DHA, Zn-EPA, and Zn-DHA-lysoPC refer to generic names for compositions comprising at least some Zn complexed with DHA, EPA, or DHA-lysoPC respectively.

### Compounds of the Invention

Aspects described herein are directed to methods of using compounds having the structure of formulae 1, below:

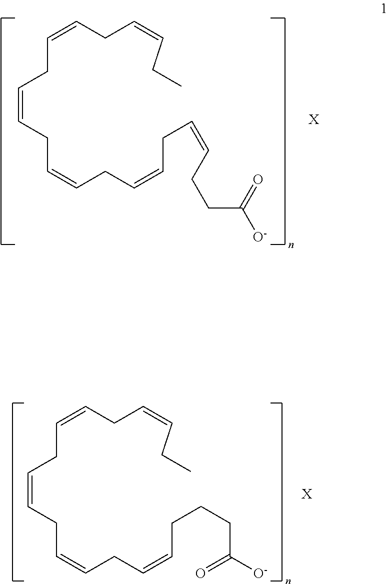

### Wherein:

X=Mg, Zn or Li
n=1 when X=mono-valent cation
n=2 when X=a di-valent cation
and solvates or polymorphs thereof.

Alternative mono-valent cation and di-valent cation complexes of derivatives of DHA and other essential fatty acids can be used in aspects described herein (e.g., mono-valent and di-valent cation complexes of epoxide derivatives of DHA, EPA, and other fatty acids). Additionally, mono-valent and di-valent cation complexes of hydroxyl derivatives can be used (e.g., diol derivatives of DHA, EPA, and other fatty acids). One aspect provides an oral dosage composition comprising Mg and DHA, at least a portion of said Mg and DHA is complexed in a salt form of Mg(DHA)₂ present in an amount equal to at least about 20 mg of Mg by weight. In other aspects, a molar ratio between said DHA and said Mg is greater than or equal to about 0.1 to 2. Generally, the molar ratio of DHA to Mg is about 2.

In some aspects, the magnesium is present in an amount greater than about 1%, 2%, 3%, 4%, 5%, 6%, 7%, 8%, 9%, 10%, 11%, 12%, 13%, 14%, or 15% by weight. In further aspects, said Mg is present in an amount greater than about 5% by weight, or in an amount greater than about 7% by weight. In some aspects, the effective daily dosage of Mg for human use can be about 50 mg to 1000 mg of elemental Mg, which corresponds to about 1.4 g to about 28 g of Mg(DHA)₂. In some aspects, the effective daily dosage of Mg for human use can be about 50 mg to 500 mg of Mg, which corresponds to about 1.4 g to about 14 g of Mg(DHA)₂. In some further aspects, the effective daily dosage of Mg for human use can be about 50 mg to 150 mg of Mg, which corresponds to about 1.4 g to about 4.2 g of Mg(DHA)₂. Similarly, compositions of Mg(EPA)₂ or Mg(DHA-LysoPC)₂ that provide a daily dosage of Mg from about 50 mg to about 500 mg, more preferably from about 50 mg to about 150 mg can be formulated according to molar ratios of 0.1 to 2 for Mg to EPA or DHA-LysoPC.

The compositions described herein generally comprise a sufficient amount of magnesium ion and DHA wherein magnesium ion or DHA may or may not be in the form of Mg(DHA)₂ in the compositions. When magnesium is not in the form of Mg(DHA)₂ but another Mg salt, the other Mg salt may be any inorganic or organic magnesium salt that is generally non-toxic. Examples of such salts include, but are not limited to, magnesium salts of chloride, sulfate, malate, acetate, oxide, lactate, citrate, gluconate, taurinate, and pidolate.

Additional aspects provide an oral dosage composition comprising Li and DHA, at least a portion of said Li complexed to said DHA as LiDHA, present in an amount equal to at least about 50 mg to 500 mg of elemental Li by weight. In other embodiments, a molar ratio between said DHA and said Li is greater than or equal to about 0.1 to 1. Generally, the molar ratio of DHA to Li is about 1. In some aspects, the effective daily dosage for human use can be about 50 mg to 500 mg of elemental Li, which corresponds to about 2.4 g to about 24 g of LiDHA. In some further aspects, the effective daily dose of Li for human use can be about 50 mg to 350 mg of elemental Li, which corresponds to about 2.4 g to about 17 g of LiDHA. Similarly, compositions of LiDHA-lysoPC or LiEPA that provide a daily dosage of elemental Li from about 50 mg to 500 mg, more preferably from 50 mg to 350 mg can be formulated according to molar ratios of 0.1 to 1 for Li to DHA-lysoPC or EPA. The compositions described herein generally comprise a sufficient amount of Li and DHA, wherein Li or DHA may or may not be in the form of LiDHA in said compositions. When Li is not in the form of LiDHA but another Li salt, the other Li salt may be any organic or inorganic Li salt that is generally non-toxic.

One aspect provides an oral dosage composition comprising Zn and DHA, at least a portion of said Zn and DHA is complexed in a salt form of Zn(DHA)₂ present in an amount equal to 5 mg by weight. In other aspects, a molar ratio between said DHA and said Zn is greater than or equal to about 0.1 to 2. Generally, the molar ratio of DHA to Zn is about 2. In some aspects, the effective daily dosage of elemental Zn for human use can be about 10 mg to 50 mg of Zn, which corresponds to about 110 mg to about 553 mg of Zn(DHA)₂. In some further aspects, the effective daily dosage of elemental Zn for human use can be about 20 mg to 30 mg of Zn, which corresponds to about 220 mg to about 332 mg of Zn(DHA)₂. Similar compositions of Zn(EPA)₂ or Zn(DHA-lysoPC)₂ that provide a daily dosage of elemental Zn from 10 mg to 50 mg, more preferably from 20 mg to 30 mg can be formulated according to molar ratios of 0.1 to 2 for Zn to EPA or DHA-lysoPC. The compositions described herein generally comprise a sufficient amount of Zn ion and DHA wherein Zn ion or DHA may or may not in the form of Zn(DHA)₂ in the compositions. When Zn is not in the form of Zn(DHA)₂ but another Zn salt, the other Zn salt may be any organic or inorganic Zn salt that is generally non-toxic.

Further aspects include the use of cationic salts e.g. magnesium salts of other fatty acids including all-cis-7,10,13-hexadecatrienoic acid, all-cis-9,12,15-octadecatrienoic acid, all-cis-6,9,12,15-octadecatetraenoic acid, all-cis-11,14,17-eicosatrienoic acid, all-cis-8,11,14,17-eicosatetraenoic acid, all-cis-5,8,11,14,17-eicosapentaenoic acid, all-cis-6,9,12,15,18-heneicosapentaenoic acid, all-cis-7,10,13,16,19-docosapentaenoic acid, all-cis-7,10,13,16,19-docosapentaenoic acid, all-cis-9,12,15,18,21-tetracosapentaenoic acid, all-cis-6,9,12,15,18,21-tetracosahexaenoic acid. Cationic salts e.g. magnesium salts of derivatives of these fatty acids including epoxides and diols are included in some aspects.

It is known that non-covalent bonding between a cation (e.g. Li, Na) and a pi-system such as a double bond is possible and further aspects include methods of using of non-covalent complexes of the fatty acids with cations e.g. Li, Na, Mg and Zn, An example of such non-covalent complex of Mg with DHA is shown in formula 2.

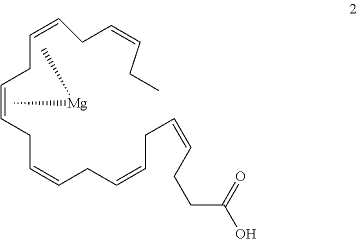

Further aspects include cationic complexes e.g. magnesium complexes of pro-drugs of the carboxylic acid moiety of the fatty acids including but not limited to esters, for example, methyl, ethyl, propylene glycol linkers, and amide pro-drugs e.g. glycoamides. Other pro-drugs of DHA include DHA-LysoPC (lysophosphatidylcholine) that is an ester of DHA that is converted to DHA in blood plasma. Cationic complexes with DHA-LysoPC may exert their beneficial effects, as claimed in some aspects, independent of conversion to DHA. Other examples of pro-drugs of carboxylic acids are exemplified in the reference Pro-drugs Biotechnology: Pharmaceutical Aspects, 2007, Volume V, Part III, 703-729. An example of LysoPC-DHA with monovalent or divalent cation is shown in formula 3, whereas LysoPC-DHA and monovalent or divalent cation non-covalent complex is shown in formula 4:

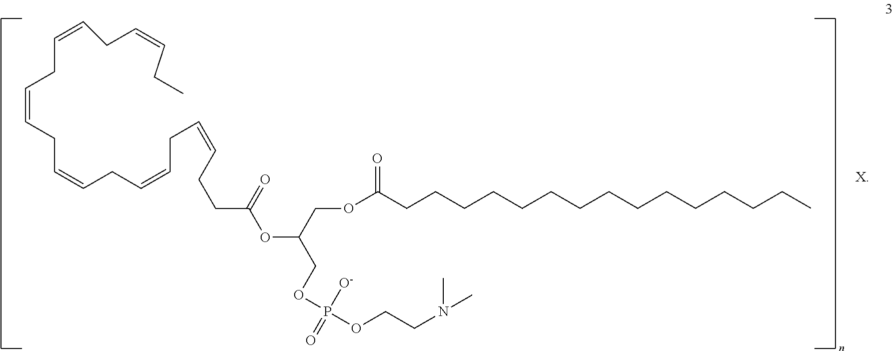

### Wherein:

X=Mg, Zn, Li
n=1 when X=mono-valent cation
n=2 when X=a di-valent cation
and solvates or polymorphs thereof

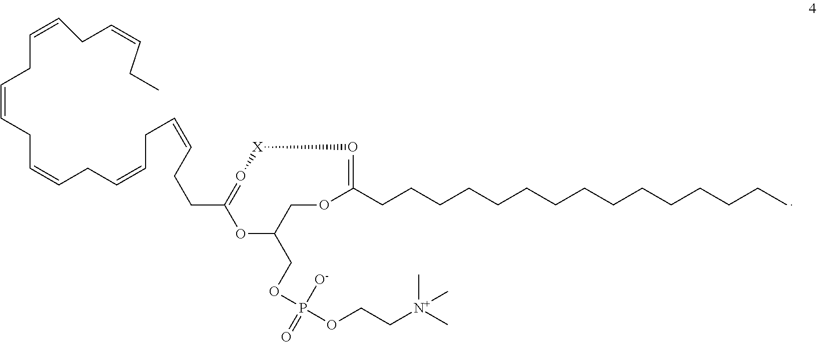

### Wherein:

X=Mg, Zn, or Li
n=1 when X=mono-valent cation
n=2 when X=a di-valent cation
and solvates or polymorphs thereof.

Magnesium-DHA or Magnesium-DHA-lysoPC is predicted to show highest bioavailability in cerebrospinal fluid (CSF) in comparison to commonly used magnesium supplements. The ability to rapidly and efficiently deliver magnesium from blood to CSF makes this compound an excellent candidate for pharmaceutical applications such as treating neurological disorders or deficiencies associated with magnesium deficit.

DHA, an un-branched fatty acid with six double bonds, all cis, is a naturally occurring compound. DHA can be isolated from its natural sources, for example, fish oil or can be chemically synthesized. In one aspect, the source of DHA for human consumption is from Market Biosciences Corporation of Columbia, Md., which has a patented system for manufacturing DHA using microalgae, which synthesize only the cis-isomer of DHA, the isomeric form safe for human consumption. See, e.g., U.S. Pat. Nos. 5,374,657, 5,492,938, 5,407,957, and 5,397,591.

Since DHA can be unstable in the presence of oxygen, anti-oxidants may optionally be added to the formulation after it is synthesized, to stabilize DHA and its conjugates. In one such method of synthesis, as mentioned in U.S. Pat. No. 6,602,902B2, the formulation is prepared in the presence of a mixture of antioxidants comprising alpha-tocopherol, dialaurylthiodipropionic acid, and ascorbic acid and held under argon in amber, sealed and stored at four degrees centrigrade. The following antioxidants may also be used singly or in combination; ascorbyl palmitate, dilaurly ascorbate, hydroquinione, butyated hydroxyanisol, sodium meta-bisulfite, t-beta carotene, x-tocopherol and astaxanthin. Similar addition of antixodants to the formulation described elsewhere may be envisioned, for example for formulation containing cations complexed with EPA, DHA-LysoPC, or fatty acids.

The magnesium-fatty acid compound may be any suitably bioavailable composition. Bioavailability of a magnesium-fatty acid compound may be determined or measured in any suitable way or using any suitable criterion. Generally, bioavailability of magnesium may refer to either the amount of magnesium that can be absorbed by a subject or the rate of absorption of magnesium by a subject or a combination of both. Further, bioavailability of magnesium may vary from subject to subject based on a variety of factors such as metabolic rate, kidney function, overall health, or other factors related to a subject, or a property of the magnesium-fatty acid compound itself, or its method of delivery. Further, the magnesium-fatty acid compound may be any suitable biologically acceptable composition.

Aspects described herein include the use of Mg-DHA, Mg-DHA-LysoPC, Mg-EPA, or magnesium compounds of fatty acids, including derivatives of fatty acids, or a mixture thereof, to increase the levels of magnesium in the cerebrospinal fluid (CSF) to near physiological levels.

One aspect provides a range of physiological beneficial concentrations of cations such as Mg, Zn, or Li to affect a desired physiological effect. For example, the amount of Mg-DHA, Mg-DHA-lysoPC-PC, or Mg-EPA provided is such that the magnesium concentrations in the CSF increase by at least about 5% to about 30% or more. Aspects provided herein describe the use of such compounds to measure their positive effects to enhance cognition in suitable animal models, known to persons of ordinary skills in the field. Such animal tests include “novel object recognition test”, “T-maze test” for spatial working memory, “Water maze test” for spatial long-term memory. Further, the effects of Mg compounds described herein can be tested for their effect on their proposed targets, such as NMDA receptor, by patch-clamp recording of neurons expressing NMDA receptor, and by measuring various biochemical markers involved in the molecular pathway related to NMDA receptor.

Aspects described herein provide methods of using Mg-DHA, Mg-DHA-lysoPC, Mg-EPA, or magnesium compounds of fatty acids to enhance cognitive function and/or prevent a decline of a subject's cognitive ability. Generally, enhancing cognitive function refers to enhancing any aspect of such a function, such as learning and memory, and acquiring, storing, retrieving, and/or using information and/or thoughts, memory. To evaluate cognition in a subject, various standardized tests such as Mini-Mental Status Exam (Folstein, 1975), PROSPER neuropsycological tests (Houx, 2002), and/or the like may be used.

In another aspects the magnesium compounds are co-administered with other cognitive enhancers, such that the efficacy contributed by the magnesium compounds allows decreased dose of the said other cognitive enhancers, thus completely or partially mitigating side effects of higher doses needed for the required efficacy by those other cognitive enhancers.

In another aspect the magnesium compounds are administered to healthy subjects to enhance their cognitive abilities.

In another aspect, the magnesium compositions are administered to provide therapeutic benefits to persons with ADHD.

In another aspect the magnesium compounds are co-administered with other cognitive enhancers, such as methylphenidate, such that the efficacy contributed by the magnesium compounds allows decreased dose of methylphenidate to achieve the desired efficacy.

In another aspect, methods of ameliorating pain in a subject in need thereof, comprising the step of administering to said subject an amount Mg-DHA, Mg-DHA-lysoPC, Mg-EPA or any variant or combination or combination or combination or combination or combination thereof, in an amount effective to have desired therapeutic effects are provided.

Further aspects provide methods of treating stroke. The methods comprise the step of administering to a subject an amount of Mg-DHA, Mg-DHA-lysoPC, Mg-EPA or any variant or combination or combination or combination or combination or combination thereof, to increase physiological concentration of magnesium to a concentration sufficient to have the desired therapeutic effects. In some instances, the concentration of magnesium is measured under a fasting condition. In other instances, the physiological concentration is serum concentration, plasma concentration, or cerebrospinal fluid concentration. In some instances, the cognitive function is short-term memory or long-term memory.

Aspects described herein provide methods of using Mg-DHA, Mg-DHA-lysoPC, Mg-EPA, magnesium compounds of fatty acids, or a combination thereof for treating depression.

In another aspect the magnesium compounds are co-administered with other antidepressants, such that the efficacy contributed by the magnesium compounds allows decreased dose of antidepressant, thus mitigating side effects of the higher doses of the said other antidepressants.

Aspects described herein provide a method of therapeutic or prophylactic treatment of zinc related disorders, comprising administering to a said subject, in need thereof, a composition of Zn-DHA, Zn-DHA-lysoPC, Zn-EPA or any variant or combination thereof in dose(s) which leads to concentrations of zinc in the brain or CNS sufficient to provide therapeutic benefits on the disease.

Aspects described herein provide methods of enhancing therapeutic efficacy of NMDA receptor modulating ions such as Mg. The said combination dosing may provide an optimal therapeutic efficacy, where a single agent does not reach that desired level. In addition, a lower dose of one or both compositions can be used, due to additive or synergistic interaction of each active ingredient.

In another aspect, methods of treating bipolar disorders are provided The method comprises the step of administering to a subject in need for a therapeutic treatment of bipolar disorder a composition of Li-DHA, Li-DHA-lysoPC, Li-EPA or any variant or combination thereof with a sufficient therapeutic dose. In another aspect, the therapeutic dosing regimen results in lithium level preferably in 0.6 to 1.2 mEq/l range in the blood. The lithium compositions described in some aspects are more bioavailable in the brain, hence require lesser amount of lithium equivalent than currently given, thus mitigating the side effects in peripheral tissues outside the nervous system.

In another aspect, lithium compounds are co-administered with other medicines for bipolar syndrome, such that the efficacy contributed by the magnesium compounds allows decreased dose of other medicines, thus mitigating side effects of their higher doses. Other medicines include, but not limited to, lamotrigine, sodium valproate and Carbamazepine.

To those skilled in the art, numerous equivalents to the specific products and processes described above will be obvious with no more than routine experimentation. Such equivalents are intended to be included within the scope of the appended claims.

### Synthesis

The magnesium, lithium and zinc salts of DHA may be prepared according to Scheme 1. Reaction of DHA with sodium hydroxide produces the sodium salt of DHA. The lithium salt can be similarly prepared by reaction of DHA with lithium hydroxide. Treatment of the sodium salt of DHA with magnesium chloride gives the magnesium slat. Alternatively reaction of the sodium salt of DHA with zinc chloride produces the zinc salt of DHA. The zinc salts can also be prepared by reaction of DHA with zinc metal or with zinc carbonate.

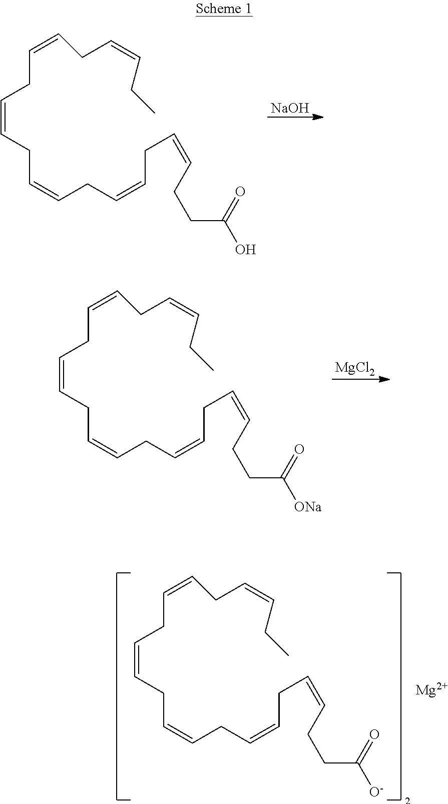

### Administration, Dosages, and Formulation

Generally, dosages and routes of administration of compounds described herein will be determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, nasal administration, perineurally or parenterally, that is, by intravenous, subcutaneous, intraperitoneal, intra-thecal, or intramuscular injection, among others, including buccal, rectal and transdermal administration. One of ordinary skill in the art may modify the formulations within the teachings of the specification to provide numerous formulations for a particular route of administration. It is also well within the ordinary skill of the art to modify the route of administration and dosage regimen of a particular composition to manage the pharmacokinetic of the present compounds for maximum beneficial effect in a patient.

On skilled in the art may adjust doses of compositions of the aspects described herein. For example, the subject would initially receive a low dose; dose would be increased if the low dose were not effective. The adjustments may be made based on efficacy or side effects or both. Compositions of some aspects can be provided in an extended or modified release form to provide active agent over a desired period of time.

In some aspects, compositions are provided as a food additive or as a medical food.

Subjects contemplated for treatment according to the methods discussed herein include humans, companion animals, laboratory animals, and the like.

In one aspect, compounds described herein may be presented in a gel cap form for oral intake by subjects in need thereof. In some other aspects, formulations may take the form of solid, semi-solid, lyophilized powder, or liquid dosage forms, such as, for example, tablets, capsules, powders, sustained-release formulations, solutions, suspensions, emulsions, suppositories, creams, ointments, lotions, aerosols, patches or the like, preferably in unit dosage forms suitable for simple administration of precise dosages.

In certain aspects, pharmaceutical compositions typically include a conventional pharmaceutical carrier or excipient and may additionally include other medicinal agents, carriers, adjuvants, additives and the like. In another aspect, the composition will be about 0.5 to 75% by weight of a compound or compounds described herein, with the remainder consisting essentially of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

In other aspects, pharmaceutical composition further comprises excipients, which enhance the permeability of active ingredient through the blood brain or blood nerve barrier. Examples of such excipients include inhibitors of P-gp or Bcrp1 or both (e.g. Elacridar), quinidine, cyclosporine, or nitric oxide producing substances, such as activators of nitric oxide synthase, bradykinin receptor agonists, and nitric oxide donors. In some other aspects, the excipients are selected from the literature to provide extended, modified, or pulsatile release of active ingredient.

Liquid compositions can be prepared by dissolving or dispersing the compounds (about 0.5% to about 20% by weight or more), and optional pharmaceutical adjuvants, in a carrier, such as, for example, aqueous saline, aqueous dextrose, glycerol, β-cyclodextrin, hydroxypropyl-β-cyclodextrin or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methylcellulose, a filler, a disintegrator, and other additives typically used in the manufacture of medical preparations.

An injectable composition for parenteral administration will typically contain the compound in a suitable intravenous solution, such as sterile physiological salt solution. The composition may also be formulated as a suspension in a lipid or phospholipid, in a liposomal suspension, or in an aqueous emulsion.

Methods for preparing such dosage forms are known or will be apparent to those skilled in the art; for example, see Remington's Pharmaceutical Sciences (17th Ed., Mack Pub. Co., 1985). The composition to be administered will contain a quantity of the selected compound in a pharmaceutically effective amount.

### EXAMPLES

Aspects described herein will be better understood by reference to the following proposed clinical study example, which is provided as exemplary, and not by way of limitation.

### Example 1

### Assay of Mg Levels in CSF

Sprague-Dawley rats are fed diet supplemented with DHA, Mg-DHA, or

Mg Citrate through oral gavage for three weeks. Cerebro-spinal fluid is collected from the rats at day 21 and magnesium levels measured using MG Flex reagent cartridge, Cat. No. DF57 provided on Dimension clinical chemistry system (SIEMENS). The MG method is a modification of the methylthymolblue (MTB) complexometric procedure described by Connerty, Lau and Briggs. The barium salt of ethylenebis (oxyethylenenitillo) tetraacetic acid (Ba-EGTA) is used to reduce interference due to calcium that also reacts with MTB. MTB is added according to the protocol supplied with Flex reagent cartridge and the amount of Mg-MTB complex formed, which is proportional to the magnesium concentration, is measured using bichromatic (600 and 510 nm) endpoint technique.

### Example 2

### Use of Mg Compositions to Enhance Mg Levels in the Cerebrospinal Fluid

The present example demonstrates the use of Mg compositions to enhance Mg levels in cerebrospinal fluid from rat brains. Young Sprague-Dawley rats weighing at least 300 g are fed various magnesium compositions by oral gavage so as to provide about 50 mg elemental Magnesium per kg per day for 3 weeks. The magnesium compositions include Mg-DHA, Mg-DHA-LysoPC and Mg-EPA. Control rats were administered equivalent molar amount of Magnesium chloride. Cerebrospinal fluid is collected at the end of the treatment, and magnesium is measured by Siemens Dimension system, based on a modification of the methylthymol blue (MTB) complexometric procedure. Rats fed with the test magnesium compositions exhibit higher CSF levels of Mg than those fed with Magnesium chloride.

### Example 3

### Use of Zn Compositions to Enhance Zn Levels in the Cerebrospinal Fluid

The present example demonstrates the use of Zn compositions to enhance Zn levels in cerebrospinal fluid from rat brains. Sprague-Dawley rats are fed various zinc compositions by oral gavage so as to provide about 1 mg elemental zinc per kg per day for 3 weeks. The zinc compositions include Zn-DHA, Zn-DHA-LysoPC and Zn-EPA. Control rats were administered equivalent molar amount of zinc gluconate. Cerebrospinal fluid is collected at the end of the treatment, and zinc is measured by atomic absorption spectroscopy. Rats fed with the test zinc compositions exhibit higher CSF levels of Zn than those fed with Zinc gluconate.

### Example 4

### Use of Li Compositions to Enhance Li Levels in the Cerebrospinal Fluid

The present example demonstrates the use of Li compositions to enhance Li levels in cerebrospinal fluid from rat brains. Sprague-Dawley rats are fed various zinc compositions by oral gavage so as to provide about 20 mg of elemental Li per Kg per day for 3 weeks. The lithium compositions include Li-DHA, Li-DHA-LysoPC and Li-EPA. Control rats were administered equivalent molar amount of Lithium carbonate. Cerebrospinal fluid is collected at the end of the treatment, and lithium is measured by atomic absorption spectroscopy. Rats fed with the test lithium compositions exhibit higher CSF levels of lithium than those fed with lithium carbonate.

### Example 5

### Use of Mg-DHA Composition to Enhance Cognitive Function

The present example demonstrates the use of Mg-DHA composition to enhance cognitive function in animals. Mg-DHA composition is provided as supplement in standard rat feed. On an average, rats consume about 50 mg/Kg/day of elemental Mg, on a feeding schedule so as to maintain 85% of its free-feeding weight. In the control group, rats are fed either DHA or magnesium citrate (Mg Citrate) supplemented diet on schedule similar to test group. After habituation to the apparatus and pre-training, each rat undergoes 4 trials of standard T-maze test (Shoji et al, J. Vis. Exp. (60), e3300, DOI: 10.3791/3300 (2012)) on day 1 or day 21 of Mg-DHA, Mg Citrate, or DHA feeding. Rats are subject to either forced alternation task or left-right discrimination task. Behaviors in the T-maze apparatus is recorded and statistically analyzed by two way repeated measures analysis of variance. Rats fed on diet supplemented with Mg-DHA show statistically significant increase in working and reference memory as compared to DHA or Mg Citrate supplemented diet fed rats. In another set of experiments, rats fed with DHA, Mg Citrate, or Mg-DHA for 21 days are subject to novel-object-location (NOL) tests. Rats fed on diet supplemented with Mg-DHA show statistically significant improvement in their ability to recognize novel objects as compared to DHA or Mg Citrate supplemented diet fed rats.

The present example demonstrates the use of Mg-DHA composition to enhance cognitive function in a human subject. Mg-DHA composition is prepared and formulated as capsules to provide a daily dose of 150 mg of elemental magnesium. The human subjects are divided into control and test groups and provided DHA, Mg Citrate (equivalent to 150 mg of elemental magnesium) or Mg-DHA capsules for 30 consecutive days. On day 1 and day 30, two cognitive tests, Montreal Cognitive Assessment and Mini-Mental State Examination (Int J Geriatr Psychiatry. 2010 February; 25(2):111-20) are administered. The subjects on Mg-DHA show statistically significant improvement over those provided with either DHA or Mg Citrate.

### Example 6

### Use of Mg-DHA-LysoPC Composition to Enhance Cognitive Function

The present example demonstrates the use of Mg-DHA-LysoPC composition to enhance cognitive function in animals. Mg-DHA composition is provided as supplement in standard rat feed. On an average, rats consume about 50 mg/Kg/day of elemental Mg, on a feeding schedule so as to maintain 85% of its free-feeding weight. In the control group, rats are fed DHA-LysoPC or Mg Citrate supplemented diet on schedule similar to test group. After habituation to the apparatus and pre-training, each rat undergoes 4 trials of standard T-maze test (Shoji et al, J. Vis. Exp. (60), e3300, DOI: 10.3791/3300 (2012)) on day 1 or day 21 of Mg-DHA-LysoPC, Mg Citrate or DHA-LysoPC feeding. Rats are subjected to either forced alternation task or left-right discrimination task. Behaviors in the T-maze apparatus is recorded and statistically analyzed by two way repeated measures analysis of variance. Rats fed on diet supplemented with Mg-DHA-LysoPC show statistically significant increase in working and reference memory as compared to DHA-LysoPC or Mg Citrate fed rats. In another set of experiments, rats fed with Mg Citrate, DHA-LysoPC, or Mg-DHA-LysoPC for 21 days are subjected to novel-object-location (NOL) tests. Rats fed with diet supplemented with Mg-DHA-LysoPC show statistically significant improvement in their ability to recognize novel objects as compared to rats fed with diet supplemented with DHA-LysoPC or Mg Citrate.

The present example demonstrates the use of Mg-DHA-LysoPC composition to enhance cognitive function in a human subject. Mg-DHA-LysoPC composition is prepared and formulated as capsules to provide a daily dose of 150 mg of elemental magnesium. The human subjects are divided into control and test groups and provided either DHA-LysoPC, Mg Citrate (equivalent to 150 mg of elemental magnesium), Mg-DHA-LysoPC capsules for 30 consecutive days. On day 1 and day 30, two cognitive tests, Montreal Cognitive Assessment and Mini-Mental State Examination (Int J Geriatr Psychiatry. 2010 February; 25(2):111-20) are administered. The subjects on Mg-DHA-LysoPC show statistically significant improvement over those provided with DHA-LysoPC or Mg Citrate.

### Example 7

### Use of Mg-EPA Composition to Enhance Cognitive Function

The present example demonstrates the use of Mg-EPA composition to enhance cognitive function in animals. Mg-EPA composition is provided as supplement in standard rat feed. On an average, rats consume about 50 mg/Kg/day of elemental Mg, on a feeding schedule so as to maintain 85% of its free-feeding weight. In the control group, rats are fed EPA or Mg Citrate supplemented diet on scheduled similar to test group. After habituation to the apparatus and pre-training, each rat undergoes 4 trials of standard T-maze test (Shoji et al, J. Vis. Exp. (60), e3300, DOI: 10.3791/3300 (2012)) on day 1 or day 21 of Mg-EPA, Mg Citrate, or EPA feeding. Rats are subjected to either forced alternation task or left-right discrimination task. Behaviors in the T-maze apparatus is recorded and statistically analyzed by two way repeated measures analysis of variance. Rats fed on diet supplemented with Mg-EPA show statistically significant increase in working and reference memory as compared to EPA, or Mg Citrate supplemented diet fed rats. In another set of experiments, rats fed with diet supplemented with EPA, Mg-EPA, or Mg Citrate for 21 days are subject to novel-object-location (NOL) tests. Rats fed with Mg-EPA supplemented diet show statistically significant improvement in their ability to recognize novel objects as compared to EPA or Mg Citrate supplemented diet fed rats.

The present example demonstrates the use of Mg-EPA composition to enhance cognitive function in a human subject. Mg-EPA composition is prepared and formulated as capsules to provide a daily dose of 150 mg of elemental magnesium. The human subjects are divided into control and test groups and provided EPA, Mg Citrate (equivalent to 150 mg elemental magnesium), or Mg-EPA capsules for 30 consecutive days. On day 1 and day 30, two cognitive tests, Montreal Cognitive Assessment and Mini-Mental State Examination (Int J Geriatr Psychiatry. 2010 February; 25(2):111-20) are administered. The subjects on Mg-EPA show statistically significant improvement over those provided with EPA or Mg Citrate.

### Example 8

### Use of Mg-DHA Composition to Provide Pain Relief

The present example demonstrates the use of Mg-DHA to provide pain relief in rodents. Rats are fed diet supplemented with Mg-DHA, Mg Citrate, or DHA. On Mg-DHA or Mg Citrate supplemented diet, rats consume, on an average, about 50 mg/Kg/day of elemental Mg, on a feeding schedule so as to maintain 85% of its free-feeding weight. In the control group, rats are fed DHA supplemented diet on schedule similar to the test group. On 0, 7, 14, and 21 days of starting the supplement diet, rats are subjected to “tail flick test” (D'Amour and Smith, 1941). The tail is dipped into a hot bath and latency of tail flick reflex is measured. Statistically significant increased latency is observed in rats fed on Mg-DHA supplemented diet as compared to Mg Citrate or DHA supplemented diet.

The present example demonstrates the use of Mg-DHA composition to provide pain relief. Patients presenting symptoms of migraine are prescribed Mg-DHA capsules that provide a daily dose of 150 mg of elemental magnesium. The symptoms of migraine are reviewed after 4 hours. Patients receiving Mg-DHA report significant pain relief on a visual analog scale (VAS) (Huskisson E C (1982). “Measurement of pain”. J. Rheumatol. 9(5): 768-9) compared to those prescribed either DHA or Mg Citrate (formulated to provide 150 mg of elemental magnesium). For each group of patients, Excedrin is prescribed as rescue medicine.

### Example 9

### Use of Mg-DHA-LysoPC Composition to Provide Pain Relief

The present example demonstrates the use of Mg-DHA-LysoPC to provide pain relief in rodents. Rats are fed diet supplemented with Mg-DHA-LysoPC, Mg Citrate, or DHA-LysoPC. On Mg-DHA-LysoPC or Mg Citrate supplemented diet, rats consume, on an average, about 50 mg/kg/day of elemental Mg, on a feeding schedule so as to maintain 85% of its free-feeding weight. In the control group, rats are fed with DHA-LysoPC supplemented diet on schedule similar to the test group. On 0, 7, 14, and 21 days of starting the supplement diet, rats are subjected to “tail flick test” (D'Amour and Smith, 1941). The tail is dipped into a hot bath and latency of tail flick reflex is measured. Statistically significant increased latency is observed in rats fed on Mg-DHA-LysoPC supplemented diet as compared to Mg Citrate or DHA-LysoPC supplemented diet.

The present example demonstrates the use of Mg-DHA-LysoPC composition to provide pain relief. Patients presenting symptoms of migraine are prescribed Mg-DHA-LysoPC capsules that provide a daily dose of 150 mg of elemental magnesium. The symptoms of migraine are reviewed after 4 hours. Patients receiving Mg-DHA-LysoPC report significant pain relief on a visual analog scale (VAS) (Huskisson E C (1982). “Measurement of pain”. J. Rheumatol. 9(5): 768-9) compared to those prescribed either DHA-LysoPC or Mg Citrate (formulated to provide 150 mg of elemental magnesium). For each group of patients, Excedrin is prescribed as rescue medicine.

### Example 10

### Use of Mg-EPA Composition to Provide Pain Relief

The present example demonstrates the use of Mg-EPA to provide pain relief in rodents. Rats are fed diet supplemented with Mg-EPA, Mg Citrate, or EPA. On Mg-DHA-LysoPC or Mg Citrate supplemented diet, rats consume, on an average, about 50 mg/kg/day of elemental Mg, on a feeding schedule so as to maintain 85% of its free-feeding weight. In the control group, rats are fed with EPA supplemented diet on schedule similar to the test group. On 0, 7, 14, and 21 days of starting the supplement diet, rats are subjected to “tail flick test” (D'Amour and Smith, 1941). The tail is dipped into a hot bath and latency of tail flick reflex is measured. Statistically significant increased latency is observed in rats fed on Mg-EPA supplemented diet as compared to Mg Citrate or EPA supplemented diet.

The present example demonstrates the use of Mg-EPA composition to provide pain relief. Patients presenting symptoms of migraine are prescribed Mg-EPA capsules composition is prepared and formulated as capsules to provide a daily dose of 150 mg of elemental magnesium. The symptoms of migraine are reviewed after 4 hours. Patients receiving Mg-EPA report significant pain relief on a visual analog scale (VAS) (Huskisson E C (1982). “Measurement of pain”. J. Rheumatol. 9(5): 768-9) compared to those prescribed either EPA or Mg Citrate (formulated to provide 150 mg of elemental magnesium). For each group of patients, Excedrin is prescribed as rescue medicine.

### Example 11

### Use of Mg-DHA Composition to Help Recovery from Stroke

The present example demonstrates the use of Mg-DHA composition to help stroke victims recover. Patients presenting early symptoms of stroke, in addition to being provided standard of care for stroke, are prescribed Mg-DHA or Mg Citrate capsules formulated to provide a daily dose of 150 mg of elemental magnesium or DHA capsules for two weeks when recovery from stroke is assessed. Patients receiving Mg-DHA show statistically significant improvement scores as measured by the Stroke Rehabilitation Assessment of Movement (STREAM), which is associated with scores on the Box and Block test, Balance Scale, Barthel Index, gait speed, and the Timed “Up & Go” Test (Daley et. al. 1997, Physiotherapy Canada; 49:269-278)

### Example 12

### Use of Mg-DHA-LysoPC Composition to Help Recovery from Stroke

The present example demonstrates the use of Mg-DHA-LysoPC composition to help stroke victims recover. Patients presenting early symptoms of stroke, in addition to being provided standard of care for stroke, are prescribed Mg-DHA-LysoPC or Mg Citrate capsules prepared and formulated to provide 150 mg of elemental magnesium, or DHA-LysoPC capsules for two weeks when recovery is assessed. Patients receiving Mg-DHA-LysoPC show statistically significant improvement scores as measured by the Stroke Rehabilitation Assessment of Movement (STREAM), which is associated with scores on the Box and Block test, Balance Scale, Barthel Index, gait speed, and the Timed “Up & Go” Test (Daley et. al. 1997, Physiotherapy Canada; 49:269-278)

### Example 13

### Use of Mg-EPA Composition to Help Recovery from Stroke

The present example demonstrates the use of Mg-EPA composition to help stroke victims recover. Patients presenting early symptoms of stroke, in addition to being provided standard of care for stroke, are prescribed Mg-EPA or Mg Citrate capsules formulated to provide 150 mg of elemental magnesium, or EPA capsules for two weeks when recovery from stroke is assessed. Patients receiving Mg-EPA show statistically significant improvement scores as measured by the Stroke Rehabilitation Assessment of Movement (STREAM), which is associated with scores on the Box and Block test, Balance Scale, Barthel Index, gait speed, and the Timed “Up & Go” Test (Daley et. al. 1997, Physiotherapy Canada; 49:269-278)

### Example 14

### Use of Mg-DHA Composition to Help Recovery from Depression

The present example demonstrates the use of Mg-DHA to ameliorate the symptoms associated with depression in rodents model of depression. Sprague-Dawley rats are fed diet supplemented with Mg-DHA, Mg Citrate, or DHA. On Mg-DHA or Mg Citrate supplemented diet, rats consume, on an average, about 50 mg/kg/day of elemental Mg, on a feeding schedule so as to maintain 85% of its free-feeding weight. In the control group, rats are fed with DHA or Mg citrate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Mg-DHA supplemented diet show significant increase in swimming and climbing behavior and reduction in immobility time as compared to rats fed with either DHA or Mg Citrate supplemented diet.

The present example demonstrates the use of Mg-DHA composition to help patients recover from depression. Patients presenting symptoms of depression, in addition to being provided standard of care for depression, are prescribed Mg-DHA or Mg Citrate capsules formulated to provide 150 mg of elemental magnesium, or DHA capsules for two weeks when recovery from depression is assessed. Patients receiving Mg-DHA show statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87)

### Example 15

### Use of Mg-DHA-LysoPC Composition to Help Recovery from Depression

The present example demonstrates the use of Mg-DHA-lysoPC to ameliorate the symptoms associated with depression in rodents model of depression. Sprague-Dawley rats are fed diet supplemented with Mg-DHA-LysoPC, Mg Citrate, or DHA-lysoPC. On Mg-DHA-LysoPC or Mg Citrate supplemented diet, rats consume, on an average, about 50 mg/Kg/day of elemental Mg, on a feeding schedule so as to maintain 85% of its free-feeding weight. In the control group, rats are fed with either DHA-lysoPC or Mg Citrate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Mg-DHA-lysoPC supplemented diet show significant increase in swimming and climbing behavior and reduction in immobility time as compared to rats fed with either DHA-lysoPC or Mg Citrate supplemented diet.

The present example demonstrates the use of Mg-DHA-LysoPC composition to help patients recover from depression. Patients presenting symptoms of depression, in addition to being provided standard of care for depression, are prescribed Mg-DHA-LysoPC or Mg Citrate capsules formulated to provide 150 mg of elemental magnesium, or DHA-LysoPC capsules for two weeks when recovery from depression is assessed. Patients receiving Mg-DHA-LysoPC show statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87)

### Example 16

### Use of Mg-EPA Composition to Help Recovery from Depression

The present example demonstrates the use of Mg-EPA to ameliorate the symptoms associated with depression in rodents model of depression. Sprague-Dawley rats are fed diet supplemented with Mg-EPA, Mg Citrate, or EPA. On Mg-EPA or Mg Citrate supplemented diet, rats consume, on an average, about 50 mg/Kg/day of elemental Mg, on a feeding schedule so as to maintain 85% of its free-feeding weight. In the control group, rats are fed with either EPA or Mg Citrate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Mg-EPA supplemented diet show significant increase in swimming and climbing behavior and reduction in immobility time as compared to rats fed with either EPA or Mg Citrate supplemented diet.

The present example demonstrates the use of Mg-EPA composition to help patients recover from depression. Patients presenting symptoms of depression, in addition to being provided standard of care for depression, are prescribed Mg-EPA or Mg Citrate capsules formulated to provide 150 mg of elemental magnesium, or EPA capsules for two weeks when recovery from depression is assessed. Patients receiving Mg-EPA score statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87)

### Example 17

### Use of Zn-DHA Composition to Enhance Cognitive Function

The present example demonstrates the use of Zn-DHA composition to enhance cognitive function in animals. Zn-DHA composition is provided as supplement in standard rat feed. On an average, rats consume about 1 mg/kg body weight/day of elemental Zn, on a feeding schedule so as to maintain 85% of its free-feeding weight. In the control group, rats are fed with either DHA or zinc gluconate supplemented diet on schedule similar to test group. After habituation to the apparatus and pre-training, each rat undergoes 4 trials of standard T-maze test (Shoji et al, J. Vis. Exp. (60), e3300, DOI: 10.3791/3300 (2012)) on day 1 or day 21 of supplement feeding. Rats are subjected to either forced alternation task or left-right discrimination task. Behaviors in the T-maze apparatus is recorded and statistically analyzed by two way repeated measures analysis of variance. Rats fed on Zn-DHA supplemented diet show statistically significant increase in working and reference memory as compared to rats fed on DHA or zinc gluconate supplemented diet. In another set of experiments, rats fed on DHA, zinc gluconate, or Zn-DHA supplemented diet for 21 days are subjected to novel-object-location (NOL) tests. Rats fed diet supplemented with Zn-DHA show statistically significant improvement in their ability to recognize novel objects as compared to rats fed with diet supplemented with DHA or zinc gluconate.

The present example demonstrates the use of Zn-DHA composition to enhance cognitive function in a human subject. Zn-DHA or zinc gluconate composition is prepared and formulated as capsules to provide 30 mg of elemental zinc. The human subjects are divided into control and test groups and provided DHA, zinc gluconate, or Zn-DHA capsules, 3 times a day for 30 consecutive days. On day 1 and day 30, two cognitive tests, Montreal Cognitive Assessment and Mini-Mental State Examination (Int J Geriatr Psychiatry. 2010 February; 25(2):111-20) are administered. The subjects on Zn-DHA supplements show statistically significant improvement over those provided with DHA or zinc gluconate.

### Example 18

### Use of Zn-DHA-LysoPC Composition to Enhance Cognitive Function

The present example demonstrates the use of Zn-DHA-LysoPC composition to enhance cognitive function in animals. Zn-DHA composition is provided as supplement in standard rat feed. On an average, rats consume about 1 mg/kg body weight/day of elemental Zn, on a feeding schedule so as to maintain 85% of its free-feeding weight. In the control group, rats are fed with either DHA-LysoPC or zinc gluconate supplemented diet on schedule similar to test group. After habituation to the apparatus and pre-training, each rat undergoes 4 trials of standard T-maze test (Shoji et al, J. Vis. Exp. (60), e3300, DOI: 10.3791/3300 (2012)) on day 1 or day 21 of supplement feeding. Rats are subjected to either forced alternation task or left-right discrimination task. Behaviors in the T-maze apparatus is recorded and statistically analyzed by two way repeated measures analysis of variance. Rats fed on Zn-DHA-LysoPC supplemented diet show statistically significant increase in working and reference memory as compared to rats fed on DHA-LysoPC or zinc gluconate supplemented diet. In another set of experiments, rats fed for 21 days on diet supplemented with DHA-LysoPC, zinc gluconate, or Zn-DHA-LysoPC are subjected to novel-object-location (NOL) tests. Rats fed with Zn-DHA-LysoPC show statistically significant improvement in their ability to recognize novel objects as compared to rats with diet supplemented with DHA or zinc gluconate.

The present example demonstrates the use of Zn-DHA-LysoPC composition to enhance cognitive function in a human subject. Zn-DHA or zinc gluconate composition is prepared and formulated as capsules to provide 30 mg of elemental zinc. The human subjects are divided into control and test groups and provided DHA-LysoPC, zinc gluconate, or Zn-DHA-LysoPC capsules, 3 times a day for 30 consecutive days. On day 1 and day 30, two cognitive tests, Montreal Cognitive Assessment and Mini-Mental State Examination (Int J Geriatr Psychiatry. 2010 February; 25(2):111-20) are administered. The subjects on Zn-DHA-LysoPC supplements show statistically significant improvement over those provided with DHA-LysoPC or zinc gluconate.

### Example 19

### Use of Zn-EPA Composition to Enhance Cognitive Function

The present example demonstrates the use of Zn-EPA composition to enhance cognitive function in animals. Zn-EPA composition is provided as supplement in standard rat feed. On an average, rats consume about 1 mg/kg body weight/day of elemental zinc, on a feeding schedule so as to maintain 85% of its free-feeding weight. In the control group, rats are fed with either EPA or zinc gluconate supplemented diet on schedule similar to test group. After habituation to the apparatus and pre-training, each rat undergoes 4 trials of standard T-maze test (Shoji et al, J. Vis. Exp. (60), e3300, DOI: 10.3791/3300 (2012)) on day 1 or day 21 of supplement feeding. Rats are subjected to either forced alternation task or left-right discrimination task. Behaviors in the T-maze apparatus is recorded and statistically analyzed by two way repeated measures analysis of variance. Rats fed on Zn-EPA supplemented diet show statistically significant increase in working and reference memory as compared to EPA or zinc gluconate supplemented diet fed rats. In another set of experiments, rats fed with EPA, zinc gluconate, or Zn-EPA for 21 days are subjected to novel-object-location (NOL) tests. Rats fed with Zn-EPA supplemented diet show statistically significant improvement in their ability to recognize novel objects as compared to rats fed on zinc gluconate or EPA supplemented diet.

The present example demonstrates the use of Zn-EPA composition to enhance cognitive function in a human subject. Zn-DHA or zinc gluconate composition is prepared and formulated as capsules to provide a daily dosage of 30 mg of elemental zinc. The human subjects are divided into control and test groups and provided EPA, zinc gluconate, or Zn-EPA capsules for 30 consecutive days. On day 1 and day 30, two cognitive tests, Montreal Cognitive Assessment and Mini-Mental State Examination (Int J Geriatr Psychiatry. 2010 February; 25(2):111-20) are administered. The subjects receiving Zn-EPA show statistically significant improvement over those receiving EPA or zinc gluconate.

### Example 20

### Use of Zn-DHA Composition to Provide Pain Relief

The present example demonstrates the use of Zn-DHA to provide pain relief in rodents. Rats are fed diet supplemented with Zn-DHA, Zn gluconate, or DHA. On Zn-DHA or Zn gluconate supplemented diet, rats consume about 1 mg/kg body weight/day of elemental zinc, on a feeding schedule so as to maintain 85% of its free-feeding weight. In the control group, rats are fed with DHA or zinc gluconate supplemented diet on schedule similar to the test group. On 0, 7, 14, and 21 days of starting the supplement diet, rats are subjected to “tail flick test” (D'Amour and Smith, 1941). The tail is dipped into a hot bath and latency of tail flick reflex is measured. Statistically significant increased latency is observed in rats fed on Zn-DHA supplemented diet as compared to rats fed on Zn-gluconate or DHA supplemented diet.

The present example demonstrates the use of Zn-DHA composition to provide pain relief. Patients presenting symptoms of migraine are prescribed Zn-DHA or zinc gluconate capsules formulated to provide a daily dosage of 30 mg of elemental zinc. The symptoms of migraine are reviewed after 4 hours. Patients receiving Zn-DHA report significant pain relief on a visual analog scale (VAS) (Huskisson E C (1982). “Measurement of pain”. J. Rheumatol. 9(5): 768-9) compared to those receiving DHA or zinc gluconate. For each group of patients, Excedrin is prescribed as rescue medicine.

### Example 21

### Use of Zn-DHA-LysoPC Composition to Provide Pain Relief

The present example demonstrates the use of Zn-DHA-LysoPC to provide pain relief in rodents. Rats are fed diet supplemented with Zn-DHA-LysoPC, Zn gluconate, or DHA-LysoPC. On Zn-DHA-LysoPC or zinc gluconate supplemented diet, rats consume about 1 mg/kg body weight/day of elemental zinc, on a feeding schedule so as to maintain 85% of its free-feeding weight. In the control group, rats are fed with DHA-LysoPC or zinc gluconate supplemented diet on schedule similar to the test group. On 0, 7, 14, and 21 days of starting the supplement diet, rats are subjected to “tail flick test” (D'Amour and Smith, 1941). The tail is dipped into a hot bath and latency of tail flick reflex is measured. Statistically significant increased latency is observed in rats fed on Zn-DHA-LysoPC supplemented diet as compared to rats fed on zinc gluconate or DHA supplemented diet.

The present example demonstrates the use of Zn-DHA-LysoPC composition to provide pain relief. Patients presenting symptoms of migraine are prescribed Zn-DHA-LysoPC or zinc gluconate formulated to provide a daily dosage of 30 mg of elemental zinc. The symptoms of migraine are reviewed after 4 hours. Patients receiving Zn-DHA-LysoPC report significant pain relief on a visual analog scale (VAS) (Huskisson E C (1982). “Measurement of pain”. J. Rheumatol. 9(5): 768-9) compared to those receiving DHA-LysoPC or zinc gluconate. For each group of patients, Excedrin is prescribed as rescue medicine.

### Example 22

### Use of Zn-EPA Composition to Provide Pain Relief

The present example demonstrates the use of Zn-EPA to provide pain relief in rodents. Rats are fed diet supplemented with Zn-EPA, zinc gluconate, or EPA. On Zn-EPA or zinc gluconate supplemented diet, rats consume about 1 mg/kg body weight/day of elemental zinc, on a feeding schedule so as to maintain 85% of its free-feeding weight. In the control group, rats are fed with EPA or zinc gluconate supplemented diet on schedule similar to the test group. On 0, 7, 14, and 21 days of starting the supplement diet, rats are subjected to “tail flick test” (D'Amour and Smith, 1941). The tail is dipped into a hot bath and latency of tail flick reflex is measured. Statistically significant increased latency is observed in rats fed on Zn-EPA supplemented diet as compared to rats fed on zinc gluconate or EPA supplemented diet.

The present example demonstrates the use of Zn-EPA composition to provide pain relief. Patients presenting symptoms of migraine are prescribed Zn-EPA capsules formulated to provide a daily dosage of 30 mg of elemental Zn. The symptoms of migraine are reviewed after 4 hours. Patients receiving Zn-EPA report significant pain relief on a visual analog scale (VAS) (Huskisson E C (1982). “Measurement of pain”. J. Rheumatol. 9(5): 768-9) compared to those prescribed either EPA or zinc gluconate. For each group of patients, Excedrin is prescribed as rescue medicine.

### Example 23

### Use of Zn-DHA Composition to Help Recovery from Stroke

The present example demonstrates the use of Zn-DHA composition to help stroke victims recover. Patients presenting early symptoms of stroke, in addition to being provided standard of care for stroke, are prescribed either Zn-DHA or zinc gluconate capsules formulated to provide a daily dosage of 30 mg of elemental Zn, or DHA capsules for two weeks when recovery from stroke is assessed. Patients receiving Zn-DHA score statistically significant improvement scores as measured by the Stroke Rehabilitation Assessment of Movement (STREAM), which is associated with scores on the Box and Block test, Balance Scale, Barthel Index, gait speed, and the Timed “Up & Go” Test (Daley et. al. 1997, Physiotherapy Canada; 49:269-278) as compared to patients receiving DHA or zinc gluconate.

### Example 24

### Use of Zn-DHA-LysoPC Composition to Help Recovery from Stroke

The present example demonstrates the use of Zn-DHA-LysoPC composition to help stroke victims recover. Patients presenting early symptoms of stroke, in addition to being provided standard of care for stroke, are prescribed either Zn-DHA-LysoPC or zinc gluconate capsules that provide a daily dosage of 30 mg of elemental Zn, or DHA-LysoPC capsules for two weeks when recovery is assessed. Patients receiving Zn-DHA-LysoPC score statistically significant improvement scores as measured by the Stroke Rehabilitation Assessment of Movement (STREAM), which is associated with scores on the Box and Block test, Balance Scale, Barthel Index, gait speed, and the Timed “Up & Go” Test (Daley et. al. 1997, Physiotherapy Canada; 49:269-278) as compared to patients receiving DHA-LysoPC or zinc gluconate.

### Example 25

### Use of Zn-EPA Composition to Help Recovery from Stroke

The present example demonstrates the use of Zn-EPA composition to help stroke victims recover. Patients presenting early symptoms of stroke, in addition to being provided standard of care for stroke, are prescribed either Zn-EPA or zinc gluconate capsules formulated to provide a daily dosage of 30 mg of elemental Zn, or EPA capsules for two weeks when recovery from stroke is assessed. Patients receiving Zn-EPA score statistically significant improvement scores as measured by the Stroke Rehabilitation Assessment of Movement (STREAM), which is associated with scores on the Box and Block test, Balance Scale, Barthel Index, gait speed, and the Timed “Up & Go” Test (Daley et. al. 1997, Physiotherapy Canada; 49:269-278) as compared to patients receiving EPA or zinc gluconate.

### Example 26

### Use of Zn-DHA Composition to Help Recovery from Depression

The present example demonstrates the use of Zn-DHA to ameliorate the symptoms associated with depression in rodents model of depression. Sprague-Dawley rats are fed diet supplemented with Zn-DHA, Zinc gluconate, or DHA. On Zn-DHA or Zinc gluconate supplemented diet, rats consume about 1 mg/kg body weight/day of elemental zinc, on a feeding schedule so as to maintain 85% of its free-feeding weight. In the control group, rats are fed with either DHA or zinc gluconate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Zn-DHA supplemented diet show significant increase in swimming and climbing behavior and reduction in immobility time as compared to rats fed with either DHA or Zn gluconate supplemented diet.

The present example demonstrates the use of Zn-DHA composition to help patients recover from depression. Patients presenting symptoms of depression, in addition to being provided standard of care for depression, are prescribed Zn-DHA or zinc gluconate capsules formulated to provide a daily dosage of 30 mg of elemental zinc, or DHA capsules for two weeks when recovery from depression is assessed. Patients receiving Zn-DHA score statistically significant improvement as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87) as compared to patients receiving DHA or zinc gluconate.

### Example 27

### Use of Zn-DHA-LysoPC Composition to Help Recovery from Depression

The present example demonstrates the use of Zn-DHA-lysoPC to ameliorate the symptoms associated with depression in rodents model of depression. Sprague-Dawley rats are fed diet supplemented with Zn-DHA-lysoPC, zinc gluconate, or DHA. On Zn-DHA-lysoPC or zinc gluconate supplemented diet, rats consume about 1 mg/kg body weight/day of elemental zinc, on a feeding schedule so as to maintain 85% of its free-feeding weight. In the control group, rats are fed with DHA-lysoPC or zinc gluconate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Zn-DHA-lysoPC supplemented diet show significant increase in swimming and climbing behavior and reduction in immobility time as compared to rats fed with either DHA-lysoPC or zinc gluconate supplemented diet.

The present example demonstrates the use of Zn-DHA-LysoPC composition to help patients recover from depression. Patients presenting symptoms of depression, in addition to being provided standard of care for depression, are prescribed Zn-DHA-LysoPC or zinc gluconate capsules formulated to provide a daily dosage of 30 mg of elemental zinc, or DHA-LysoPC capsules for two weeks when recovery from depression is assessed. Patients receiving Zn-DHA-LysoPC scored statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87) as compared to patients receiving zinc gluconate or DHA-LysoPC

### Example 28

### Use of Zn-EPA Composition to Help Recovery from Depression

The present example demonstrates the use of Zn-EPA to ameliorate the symptoms associated with depression in rodents model of depression. Sprague-Dawley rats are fed diet supplemented with Zn-EPA, zinc gluconate, or EPA. On Zn-EPA or zinc gluconate supplemented diet, rats consume about 1 mg/kg body weight/day of elemental zinc, on a feeding schedule so as to maintain 85% of its free-feeding weight. In the control group, rats are fed with EPA or zinc gluconate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Zn-EPA supplemented diet show significant increase in swimming and climbing behavior and reduction in immobility time as compared to rats fed with either EPA or Zn gluconate supplemented diet.

The present example demonstrates the use of Zn-EPA composition to help patients recover from depression. Patients presenting symptoms of depression, in addition to being provided standard of care for depression, are prescribed Zn-EPA or zinc gluconate capsules that provide a daily dosage of 30 mg of elemental Zn, or EPA capsules for two weeks when recovery from depression is assessed. Patients receiving Zn-EPA score statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87) as compared to patients receiving zinc gluconate or EPA.

### Example 29

### Use of Li-DHA Composition to Help Recovery from Depression

The present example demonstrates the use of Li-DHA to ameliorate the symptoms associated with depression in rodents model of depression. Sprague-Dawley rats are fed diet supplemented with Li-DHA, lithium carbonate, or DHA. On Li-DHA or lithium carbonate supplemented diet, rats consume, on an average, about 15.5 mg/Kg/day of elemental lithium, on a feeding schedule so as to maintain 85% of its free-feeding weight. In the control group, rats are fed with DHA or lithium carbonate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Li-DHA supplemented diet show significant increase in swimming and climbing behavior and reduction in immobility time as compared to rats fed with either DHA or lithium carbonate supplemented diet.

The present example demonstrates the use of Li-DHA composition to help patients recover from depression. Patients presenting symptoms of depression, in addition to being provided standard of care for depression, are prescribed either Li-DHA or lithium carbonate capsules that provide 113 mg of elemental Li, or DHA capsules, 3 to 4 times a day, for two weeks when recovery from depression is assessed. Patients receiving Li-DHA score statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87) as compared to patients receiving lithium carbonate or DHA.

### Example 30

### Use of Li-DHA-LysoPC Composition to Help Recovery from Depression

The present example demonstrates the use of Li-DHA-lysoPC to ameliorate the symptoms associated with depression in rodents model of depression. Sprague-Dawley rats are fed diet supplemented with Li-DHA-lysoPC, lithium carbonate, or DHA. On Li-DHA-lysoPC or lithium carbonate supplemented diet, rats consume, on an average, about 15.5 mg/Kg/day of elemental lithium, on a feeding schedule so as to maintain 85% of its free-feeding weight. In the control group, rats are fed with DHA-lysoPC or lithium carbonate supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Li-DHA-lysoPC supplemented diet show significant increase in swimming and climbing behavior and reduction in immobility time as compared to rats fed with either DHA-lysoPC or lithium carbonate supplemented diet.

The present example demonstrates the use of Li-DHA-LysoPC composition to help patients recover from depression. Patients presenting symptoms of depression, in addition to being provided standard of care for depression, are prescribed Li-DHA-LysoPC or lithium carbonate capsules formulated to provide 113 mg/Kg/day of elemental Li or DHA-LysoPC capsules, 3 to 4 times a day, for two weeks when recovery from depression is assessed. Patients receiving Li-DHA-LysoPC show statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87) as compared to patients receiving DHA-LysoPC or lithium carbonate.

### Example 31

### Use of Li-EPA Composition to Help Recovery from Depression

The present example demonstrates the use of Li-EPA to ameliorate the symptoms associated with depression in rodents model of depression. Sprague-Dawley rats are fed diet supplemented with Li-EPA, lithium carbonate, or EPA. On Li-EPA or lithium carbonate supplemented diet, rats consume, on an average, about 15.5 mg/Kg/day of elemental Li, on a feeding schedule so as to maintain 85% of its free-feeding weight. In the control group, rats are fed with EPA supplemented diet on schedule similar to the test group. On 0, 7, 14, and 21 days of starting the supplement diet, rats are subjected to “Forced Swim Test”, wherein rats are forced to swim in a deep cylinder filled with tepid water for 5 minutes (Slattery and Cryan, Nature Protocols. 2012; 7(6): 1009-1014). Various established parameters of antidepressant activity including reduced immobility time, increased swimming, and increased climbing behavior are quantitated to assess the antidepressant activity. Rats fed with Li-EPA supplemented diet show significant increase in swimming and climbing behavior and reduction in immobility time as compared to rats fed with either EPA or lithium carbonate supplemented diet.

The present example demonstrates the use of Li-EPA composition to help patients recover from depression. Patients presenting symptoms of depression, in addition to being provided standard of care for depression, are prescribed Li-EPA or lithium carbonate capsules formulated to provide 113 mg of elemental Li, or EPA capsules, 3 to 4 times a day, for two weeks when recovery from depression is assessed. Patients receiving Li-EPA score statistically significant improvement scores as measured by the Hamilton Depression Rating scale (Hamilton M: A rating scale for depression (J Neurol Neurosurg Psychiatry 1960; 23:56-62) and the Beck Depression Inventory (Beck A T: Beck Depression Inventory, in Test Critiques, vol II. Edited by Deyser D J, Sweetland R C. Kansas City, Mo., Test Corporation of America, 1985, pp 83-87) as compared to patients receiving lithium carbonate or EPA.

### Example 32

### Use of Li-DHA Composition to Treat Bipolar Disorder

The present example demonstrates the use of Li-DHA to ameliorate the symptoms associated with bipolar disorder in rodents model of bipolar disorder. The model used in this study is established according to protocol described by Mavrikaki et. al. (Int. J. Neuropsychopharm. 2012). C57BL/6J mice are fed diet supplemented with Li-DHA, lithium chloride, or DHA. On Li-DHA or lithium chloride supplemented diet, mice consume, on an average, about 15.5 mg/Kg/day of elemental lithium, on a feeding schedule so as to maintain 85% of its free-feeding weight. In the control group, mice are fed with DHA or lithium chloride supplemented diet on schedule similar to the test group. On 0, 7, 14, and 21 days of starting the supplement diet, mice are injected with 0.5 mg/Kg d-amphetamine and observed for hyperlocomotion using automated video recording device. The data is analyzed for hyperlocomotion where mice with diet supplemented with Li-DHA show lower levels of induced hyperlocomotion as compared to mice fed on diet supplemented with DHA or lithium chloride.

The present example demonstrates the use of Li-DHA composition to treat acute symptoms of bipolar disorder in rat model of bipolar disorders. C57BL/6J mice are injected with d-amphetamine (0.5 mg/Kg) and increased locomotion is measured. Fifteen minutes later, Li-DHA, or lithium chloride dose is injected to deliver 15.5 mg/Kg elemental lithium. In control group, DHA is injected in amounts similar to that found in Li-DHA composition. Subsequently, the mice are observed for hyperlocomotion for the next 120 minutes using automated video recording device. The data is analyzed for hyperlocomotion where mice injected with Li-DHA show lower levels of induced hyperlocomotion as compared to DHA or lithium chloride injected mice.

The present example demonstrates the use of Li-DHA composition to treat bipolar disorder in human subjects. Patients presenting symptoms of bipolar disorder, in addition to being provided standard of care for bipolar disorder, are prescribed either Li-DHA capsules or lithium carbonate composition formulated to provide 113 mg of elemental Li, or DHA capsules, for 3 to 4 times a day, for two weeks when depression and mania is assessed. Patients receiving Li-DHA show statistically significant improvement scores as compared to patients receiving lithium carbonate or DHA. The scores are measured by the Hamilton Depression Rating scale for depression (Hamilton M: A rating scale for depression (J Neurol Neurosurg Psychiatry 1960; 23:56-62), Beck Depression Inventory for depressed mood (Beck A T: Beck Depression Inventory, in Test Critiques, vol II. Edited by Deyser D J, Sweetland R C. Kansas City, Mo., Test Corporation of America, 1985, pp 83-87) and Young Mania Rating Scale (YMRS) as a clinician-administered instrument for mania (Br. J. Psychiatry 1978. 133 (5): 429-35).

### Example 33

### Use of Li-DHA-LysoPC Composition to Treat Bipolar Disorder

The present example demonstrates the use of Li-DHA-lysoPC to ameliorate the symptoms associated with bipolar disorder in rodents model of bipolar disorder. The model used in this study is established according to protocol described by Mavrikaki et. al. (Int. J. Neuropsychopharm. 2012). C57BL/6J mice are fed diet supplemented with Li-DHA-lysoPC, lithium chloride, or DHA-lysoPC. On Li-DHA-lysoPC or lithium chloride supplemented diet, mice consume, on an average, about 15.5 mg/Kg/day of elemental lithium, on a feeding schedule so as to maintain 85% of its free-feeding weight. In the control group, mice are fed with DHA-lysoPC or lithium chloride supplemented diet on schedule similar to the test group. On 0, 7, 14, and 21 days of starting the supplement diet, mice are injected with 0.5 mg/Kg d-amphetamine and observed for hyperlocomotion using automated video recording device. The data is analyzed for hyperlocomotion where mice with diet supplemented with Li-DHA-lysoPC show lower levels of induced hyperlocomotion as compared to mice fed on diet supplemented with DHA-lysoPC or lithium carbonate.

The present example demonstrates the use of Li-DHA-lysoPC composition to treat acute symptoms of bipolar disorder in rat model of bipolar disorders. C57BL/6J mice are injected with d-amphetamine (0.5 mg/Kg) and increased locomotion is measured. Fifteen minutes later, Li-DHA-lysoPC, or lithium chloride dose is injected to deliver 15.5 mg/Kg elemental lithium. In control group, DHA-lysoPC is injected in amounts similar to that found in Li-DHA-lysoPC composition. Subsequently, the mice are observed for hyperlocomotion for the next 120 minutes using automated video recording device. The data is analyzed for hyperlocomotion where mice injected with Li-DHA-lysoPC show lower levels of induced hyperlocomotion as compared to DHA-lysoPC or lithium chloride injected mice.

The present example demonstrates the use of Li-DHA-LysoPC composition to treat bipolar disorder in human subjects. Patients presenting symptoms of bipolar disorder, in addition to being provided standard of care for bipolar disorder, are prescribed Li-DHA-LysoPC capsules or lithium carbonate composition formulated to provide 113 mg of elemental Li, or DHA capsules, for three to four times a day, for two weeks when depression and mania is assessed. Patients receiving Li-DHA-LysoPC score statistically significant improvement scores as compared to patients receiving lithium carbonate of DHA-LysoPC. The scores are measured by the Hamilton Depression Rating scale for depression (Hamilton M: A rating scale for depression (J Neurol Neurosurg Psychiatry 1960; 23:56-62), Beck Depression Inventory for depressed mood (Beck A T: Beck Depression Inventory, in Test Critiques, vol II. Edited by Deyser D J, Sweetland R C. Kansas City, Mo., Test Corporation of America, 1985, pp 83-87) and Young Mania Rating Scale (YMRS) as a clinician-administered instrument for mania (Br. J. Psychiatry 1978. 133 (5): 429-35)

### Example 34

### Use of Li-EPA Composition to Treat Bipolar Disorder

The present example demonstrates the use of Li-EPA to ameliorate the symptoms associated with bipolar disorder in rodents model of bipolar disorder. The model used in this study is established according to protocol described by Mavrikaki et. al. (Int. J. Neuropsychopharm. 2012). C57BL/6J mice are fed diet supplemented with Li-EPA, lithium chloride, or EPA. On Li-EPA or lithium chloride supplemented diet, mice consume, on an average, about 15.5 mg/Kg/day of elemental lithium, on a feeding schedule so as to maintain 85% of its free-feeding weight. In the control group, mice are fed with EPA supplemented diet on schedule similar to the test group. On 0, 7, 14, and 21 days of starting the supplement diet, mice are injected with 0.5 mg/Kg d-amphetamine and observed for hyperlocomotion using automated video recording device. The data is analyzed for hyperlocomotion where mice with diet supplemented with Li-EPA show lower levels of induced hyperlocomotion as compared to mice fed on diet supplemented with EPA or lithium chloride

The present example demonstrates the use of Li-EPA composition to treat acute symptoms of bipolar disorder in mice model of bipolar disorders. C57BL/6J mice are injected with d-amphetamine (0.5 mg/Kg) and increased locomotion is measured. Fifteen minutes later, Li-EPA, or lithium chloride dose is injected to deliver 15.5 mg/Kg elemental lithium. In control group, EPA is injected in amounts similar to that found in Li-EPA composition. Subsequently, the mice are observed for hyperlocomotion for the next 120 minutes using automated video recording device. The data is analyzed for hyperlocomotion where mice injected with Li-EPA show lower levels of induced hyperlocomotion as compared to EPA or lithium chloride injected mice.

The present example demonstrates the use of Li-EPA composition to treat bipolar disorder in human subjects. Patients presenting symptoms of bipolar disorder, in addition to being provided standard of care for bipolar disorder, are prescribed either Li-EPA capsules or lithium carbonate composition formulated to provide 113 mg of elemental Li or EPA capsules, three to four times a day, for two weeks when depression and mania is assessed. Patients receiving Li-EPA show statistically significant improvement scores as compared to patients receiving lithium carbonate or EPA. The scores are measured by the Hamilton Depression Rating scale for depression (Hamilton M: A rating scale for depression (J Neurol Neurosurg Psychiatry 1960; 23:56-62), Beck Depression Inventory for depressed mood (Beck A T: Beck Depression Inventory, in Test Critiques, vol II. Edited by Deyser D J, Sweetland R C. Kansas City, Mo., Test Corporation of America, 1985, pp 83-87) and Young Mania Rating Scale (YMRS) as a clinician-administered instrument for mania (Br. J. Psychiatry 1978. 133 (5): 429-35).

### REFERENCES CITED

U.S. Patent documents

| | | |
|---|---|---|
| US 2008/0269327 A1 | October 2008 | Liu et al. |
| US 2008/0249178 A1 | October 2008 | Liu et al. |
| US 2008/0248137 A1 | October 2008 | Liu et al. |
| US 2008/0248100 A1 | October 2008 | Liu et al. |
| US 2008/0249169 A1 | October 2008 | Liu et al. |
| US 2008/0249170 A1 | October 2008 | Liu et al. |
| US 2011/0020443 A1 | January 2011 | Liu et al. |
| U.S. Pat. No. 5,604,198 | February 1997 | Poduslo et al. |
| U.S. Pat. No. 5,268,164 | December 1993 | Kozarich et al. |
| U.S. Pat. No. 7,012,061 | March 2006 | Reiss et al. |

### Other Publications

1. Slutsky, et al. Enhancement of learning and memory by elevating brain magnesium. Neuron. January 2010; 65(2): 165-177
2. Lagarde, et al. Lysophosphatidylcholine as a preferred carrier form of docosahexaenoic acid to the brain. J. Mol. NeuroSci. 2001; 16(2-3): 201-204
3. Baldessarini, et al. Fatty acid derivatives of clozapine: prolonged antidopaminergic activity of docosahexaenoylclozapine in the rat. Neuropsychopharmacology. 2001; January; 24(1): 55-65
4. Altamura, et al. Mood stabilizers for patients with bipolar disorder: the state of the art. Expert Rev. Neurother. 2011; January; 11(1): 85-99
5. Plenge, et al. 24-hour lithium concentration in human brain studied by Li-7 magnetic resonance spectroscopy. Biol. Psychiatry. 1994; 36(8): 511-516
6. Anesth. Analg. 1996, 83: pp 123-1228
7. Landfield, et al. Chronically elevating plasma Mg2+ improves hippocampal frequency potentiation and reversal learning in aged and young rats. Brain Res. Nov. 19, 1984; 322(1): 167-71
8. Slutsky, et al. Enhancement of synaptic plasticity through chronically reduced Ca+ flux during uncorrelated activity. Neuron. Dec. 2, 2004; 44(5): 835-49.
9. Tang, et al. Genetic enhancement of learning and memory in mice. Nature. Sep. 2, 1990; 401(6748)
10. Tramer, et al. Magnesium Bier's block for treatment of chronic limb pain: a randomized, double-blind, cross-over study. Pain. 2002; September 1999(1-2):235-41
11. Brill, et al. Efficacy of intravenous magnesium in neuropathic pain. Br. J. Anaesth. 2002; 89 (5): 711-714
12. Levaux, et al. Effect of intra-operative magnesium sulphate on pain relief and patient comfort after major lumbar orthopaedic surgery. Anaesthesia 2003; 58:131-135
13. Muir. Therapeutic potential of magnesium in the treatment of acute stroke. J Stroke Cerebrovascular diseases. December 2000; 9 (6) 257-267
14. Wang, et al. Magnesium sulfate and numesulide have synergistic effects on rescuing brain damage after transient focal ischemia. J Neurotrauma 2012; 29(7): 1518-1529.
15. Euser, et al. Magnesium sulfate for the treatment of Eclampsia. Stroke 2009; 40:1169-1175.
16. In search of clinical neuroprotection after brain ischemia: The case for mild hypothermia (35° C.) and Magnesium. Stroke 2009; 40: 2236-2240
17. Sun-Edelstein, et al. Role of magnesium in the pathogenesis and treatment of migraine. Expert Rev. Neurothera. March 2009; 9(3): 369-379
18. Vink, et al. Magnesium in acute and chronic brain injury: an update. Magnesium Research. September 2009: 22(3) 12^{th} International Magnesium symposium.
19. Eby, et al. Magnesium for treatment-resistant depression: a review and hypothesis. Medical Hypothesis. 2010 April; 74(4): 649-60
20. Eby, et al. Rapid recovery from major depression using magnesium treatment. Medical Hypotheses. 2006; 67(2): 362-70
21. Maylor, et al. Effects of zinc supplementation on cognitive function in healthy middle aged and older adults: the ZENITH study. Br. J. Nutr. 2006; 96(4): 752-760.
22. Cope, et al. Use of zinc as a treatment for traumatic brain injury in the rat. Neurorehabil Neural Repair. September 2012; 26(7): 907-913
23. Nozaki, et al. Zinc alleviates pain through high-affinity binding to the NMDA receptor NR2A subunit. Nat Neurosci. 2011; 14: 1017-1022
24. Aquilani, et al. Normalization of zinc intake enhances neurological retrieval of patients suffering from ischemic strokes. Nutr. Neurosci. 2009; 12(5): 219-25.
25. Cope, et al. Role of zinc in the development and treatment of mood disorders. Curr Opin Clin Nutr Metab Care. 2010; 13:685-689.
26. Nowak, et al. Effect of zinc supplementation on antidepressant therapy in unipolar depression: a preliminary placebo-controlled study. Pol J. Pharmacol. 2003; 55:1143-1147.
27. Levenson C. Zinc, the new antidepressant? Nutr Rev. 2006; 64:39-42.
28. Watase K et al. Lithium therapy improves neurological function and hippocampal dendritic arborization in a spinocerebellar ataxia type 1 mouse model. PloS Med. 4(5): e182. Doi: 10.1371/journal.pmed.0040182.
29. Severus, et al. What is the optimal serum lithium level in the long-term treatment of bipolar disorder—a review? Bipolar Disorder. 2008; 10:231-237.
30. Bauer, et al. Lithium augmentation in treatment-resistant depression: meta-analysis of placebo-controlled studies. J Clin Psychopharmacol. 1999 October; 19(5):427-434.
31. D'Amour and Smith. A method of determining loss of pain sensation. J Pharmacol Exp Ther 72:74-79.
32. Slattery and Cryan. Using the rat forced swim test to assess anti-depressant-like activity in rodents. Nature Protocols. 2012; 7(6): 1009-1014.
33. Mavrikaki et al. Efficacy of the atypical antipsychotic aripiprazole in d-amphetamine-based preclinical models of mania. Int J Neurophsychopharmacology 2010; 13: 541-548
34. Gould et al. Strain differences in lithium attenuation of d-amphetamine-induced hyperlocomotion: A mouse model for the genetics of clinical response to lithium. Neuropsychopharmacology 2007; 32: 1321-1333
35. Jeffrey and Summerfield. Assessment of the blood-brain barrier in CNS drug discovery. Neurobiology of Disease 2012; 37: 33-37
36. Di et al. Demystifying brain penetration in central nervous system drug discovery. J Med Chem. 2012; dx.doi.org/10.1021/jm30129f1
37. Minocha et al. Co-administration strategy to enhance brain accumulation of vandetanib by modulating P-glycoprotein (P-gp/Abcb1) and breast cancer resistance protein (Bcrp1/Abcg2) mediated efflux with m-TOR inhibitors. Int. J. Pharm. 2012; 434: 306-314
38. Liu et al. Progress in brain penetration evaluation in drug discovery and development. The J of Pharmac and Exp. Thera. 2008; 325(2) 349-356
39. Minocha et al. Enhanced brain accumulation of pazopanib by modulating P-gp and Bcrp1 mediated efflux with canertinib or erlotinib. Int. J. Pharm. 2012; 436: 127-134
40. Connerty et al. Spectrophotometric determination of magnesium by use of methylthymol blue. Clin Chem. 1971; 17: 661-667

I claim:

1. A pharmaceutical composition comprising magnesium complexed with one or more fatty acids selected from a group consisting of DHA, DHA-LysoPC and EPA, and a pharmaceutically acceptable carrier.

2. A pharmaceutical composition according to claim 1, further comprising an antioxidant.

3. A method of increasing the concentration of magnesium in cerebrospinal fluid comprising providing the composition of claim 1 to a mammal, wherein the concentration of magnesium in cerebrospinal fluid is increased by at least about 5%.

4. A method of enhancing cognitive function comprising administering a composition of claim 1 to a subject in an amount that is effective to enhance cognitive function.

5. A method of ameliorating pain comprising administering a composition of claim 1 to a subject in an amount effective to ameliorate pain.

6. A method of ameliorating depression comprising administering a composition of claim 1 in an amount effective to ameliorate depression.

7. A pharmaceutical composition comprising zinc complexed with one or more fatty acids selected from a group consisting of DHA, DHA-lysoPC and EPA, and a pharmaceutically acceptable carrier.

8. A pharmaceutical composition according to claim 7, further comprising an antioxidant.

9. A method of increasing the concentration of zinc in cerebrospinal fluid comprising providing the composition of claim 7 to a mammal, wherein the concentration of zinc in cerebrospinal fluid is increased by at least about 5%.

10. A method of enhancing cognitive function comprising administering a composition of claim 7 to a subject in an amount that is effective to enhance cognitive function.

11. A method of ameliorating pain comprising administering a composition of claim 7 to a subject in an amount effective to ameliorate pain.

12. A method of ameliorating depression comprising administering a composition of claim 7 in an amount effective to ameliorate depression.

13. A pharmaceutical composition comprising lithium complexed with one or more fatty acids selected from a group consisting of DHA, DHA-lysoPC and EPA, and a pharmaceutically acceptable carrier.

14. A pharmaceutical composition according to claim 13, further comprising an antioxidant.

15. A method of increasing the concentration of lithium in cerebrospinal fluid comprising providing the composition of claim 13 to a mammal, wherein the concentration of lithium in cerebrospinal fluid is increased by at least about 5%.

16. A method of ameliorating symptoms of bipolar disorder comprising administering a composition of claim 13 in an amount effective to ameliorate symptoms of bipolar.
